(19) United States
(12) Patent Application Publication
(10) Pub. No.: US 2026/0322879 A1
(43) Pub. Date: Oct. 15, 2026

(54) COMPUTER-IMPLEMENTED SYSTEM AND METHOD FOR INTEGRATED MONITORING OF HAZARDOUS ACTIVITIES IN CONFINED SPACES AND NON-TRANSITORY, COMPUTER-READABLE STORAGE MEDIUM

(71) Applicants: PETRÓLEO BRASILEIRO S.A. – PETROBRAS, Rio de Janeiro (BR); PIX FORCE TECNOLOGIA LTDA – EPP, Porto Alegre (BR)

(72) Inventors: Flávio Alves Dos Santos, Porto Alegre (BR); Carlos Frederico Mendes Gomes, Porto Alegre (BR); Luciane Calabria, Porto Alegre (BR); Eric Macedo Cabral, Porto Alegre (BR); Pablo Figueiredo Alberto, Porto Alegre (BR); Renato Forni, São José dos Campos (BR); Flavia Manica Siviero, Porto Alegre (BR); Priscila Dias Da Silva, Porto Alegre (BR)

(21) Appl. No.: 19/638,177

(22) Filed: Apr. 3, 2026

(30) Foreign Application Priority Data
Apr. 11, 2025 (BR) 1020250071827

(51) Int. Cl. H04L 67/12; G01N 33/00; H04L 12/40; H04L 61/103; H04L 67/02

(52) U.S. Cl. CPC H04L 67/12; G01N 33/0036; H04L 12/40; H04L 61/103; H04L 67/02; H04L 2012/40228

(57) ABSTRACT

The present invention relates to a system and method for integrated monitoring of hazardous activities in confined spaces, focusing on safety and operational efficiency, which allows for real-time monitoring. The system uses devices available on the market for collection of data. This includes the use of environmental sensing, cameras, and wearable devices and enables real-time monitoring both locally and remotely. Furthermore, the system is capable of issuing automatic alerts, identifying non-conformities, and providing detailed reports, correlating data to predict accidents and improve the worker's safety. The system further integrates a cloud-based solution for data storage and management, allowing future access for audits and forensic analysis. The combination of artificial intelligence with technical modularity offers an efficient approach to overcoming challenges typical of confined spaces, including restricted communication, limited monitoring, and response to emergencies. Therefore, the proposed solution promotes greater safety, regulatory compliance, and incident prevention in various industrial fields.

### CROSS REFERENCE TO RELATED APPLICATIONS

The present application claims priority to BR Pat. App. No. 1020250071827 filed on Apr. 11, 2025, which is hereby incorporated by reference in its entirety.

### FIELD OF THE INVENTION

The present invention falls within the technical field of oil and gas. In particular, it refers to data evaluation systems and methods; more specifically, it refers to safety solutions with control installations that require the simultaneous use of two or more body parts, and refers to a computer-implemented system and method for integrated monitoring of hazardous activities in confined spaces using cameras, environmental sensing data, and wearable devices.

### BACKGROUND OF THE INVENTION

Activities performed in confined spaces, such as tunnels or tanks, are challenging tasks that present considerable risks, including extreme temperatures and potentially toxic atmospheres. Safety is of paramount importance, requiring adequate protective equipment, rigorous training, and constant vigilance. Technologies such as multisensory systems, when combined with artificial intelligence (AI), can play a significant role in improving the monitoring of these activities.

However, it is not easy to supervise hazardous activities due to the diversity of devices available on the market, each with its own communication interface. The lack of an integration between such devices makes monitoring inefficient and data management in hazardous work sites, such as confined spaces and high-risk areas, a challenge.

Confined space monitoring is generally carried out in compliance with the basic requirements of NR-33—Health and Safety in Confined Spaces, which mandates the presence of an attendant controlling access to the confined space, the use of a multi-gas sensor by authorized workers, and radio for external communication with the attendant. However, such monitoring methodology tends to be more reactive and involves assessing risk scenarios during the execution of the activities.

Another challenge is the lack of other relevant data such as images and person's vital signs. Some of the main challenges and risks associated with this process are the reliance on attendants, since the monitoring of confined spaces is often exclusively dependent on attendants, who are responsible for controlling the entry and exit of workers. This dependency places a great responsibility on the attendant, who lack adequate means to monitor workers' activities inside the confined space.

Communication restraints represent one of the main challenges in monitoring confined spaces. Typically, attendants rely exclusively on radios as a means of communication, which are subject to failure due to interference, signal issues, or equipment malfunction. This creates a significant barrier to an effective communication between the attendant and workers inside the confined space, in particular in emergencies or when assistance is required.

Furthermore, there is no internal monitoring, due to the absence of an internal monitoring system within the confined space, which results in lack of visibility into the workers' activities. This increases the risk of accidents, injuries, or undetected emergencies, since there is no way to monitor and react appropriately to events taking place inside the confined space.

There is also the risk of undetected incidents, since the lack of internal monitoring means that incidents such as falls, chemical leaks, or worker health issues can go unnoticed until they become serious. This increases the potential for harm to the health and safety of the workers involved.

Moreover, there is some difficulty in responding to emergencies, such as in the case of an emergency inside the confined space (leaks of toxic substances or lack of oxygen), which, due to a lack of internal monitoring and effective communication, prevents a quick and effective response to risky scenarios. This results in increased response times and the potential for harm to the workers involved.

Therefore, it is noted that confined space monitoring faces significant challenges and presents considerable risks due to a lack of adequate resources for communication, internal monitoring, and emergency response. This lack of resources jeopardizes the safety and well-being of workers involved in these hazardous activities.

In view of the foregoing, in order to solve the limitations and technical issues described above, the present invention proposes a computer-implemented method and a type of unifying platform (system) that integrates various devices available on the market. The platform provides a single interface for monitoring and managing data, simplifying the process for operators and improving monitoring efficiency.

Also, the platform combines intelligence from the collected data, enabling the early detection of anomalies and more informed real-time decision-making. The scope of this solution is broad and encompasses any work environment where monitoring of hazardous activities is required.

This includes industries such as oil and gas, construction, mining, manufacturing, among others. The platform can be adapted to meet the specific requirements of each sector, thus providing a flexible and comprehensive solution for security monitoring.

### STATE OF THE ART

To assess the state of the art, existing patents aligned with the topic of monitoring hazardous activities in confined spaces were considered, and four documents were found: U.S. Pat. No. 8,493,223B2, CN103513636B, U.S. Pat. Nos. 10,674,965B2 and 11,954,751B2. The main distinguishing features of each invention were analyzed both in comparison with the computer-implemented system and method for integrated monitoring of risk activities in confined spaces, and with the combination of techniques in relation to the proposed invention.

U.S. Pat. No. 8,493,223B2 and CN103513636B deal with monitoring systems in confined spaces, but they provide distinct approaches and significant limitations, both in integration and adaptation, in particular when compared to the new invention. Document U.S. Pat. No. 8,493,223B2 describes a safety monitoring system that relies on an umbilical cable for data transmission between the working module and the monitoring unit. This approach imposes significant physical constraints and limits the system's mobility. Furthermore, U.S. Pat. No. 8,493,223B2 makes use of a fixed working module with specific components, such as gas sensors and cameras, which need to be directly connected to the monitoring unit via cable.

CN103513636B proposes a system in which environmental sensors, operating elements, and the radio unit are integrated in a single box. To combine this concept with U.S. Pat. No. 8,493,223B2, which uses fixed, wired modules, it would be required to redesign the sensors and equipment layout, resulting in the creation of a hybrid module unifying both approaches. This process would require not only physical adaptation, such as the development of connectors and communication interfaces, but also adjustments to the system's operating logic, since the sensors of CN103513636B are designed to operate within an integrated structure, while those of U.S. Pat. No. 8,493,223B2 operate in a distributed manner and are wired (connected via cable).

In contrast, the present invention eliminates this need for complex integration by adopting a modular and flexible approach. The system may initially operate using only cameras, without requiring gas sensors or wearable devices, thereby allowing implementation to occur gradually and in accordance with the specific needs of each operation. This represents a significant advantage in terms of time and cost, since there is no need for complex hardware settings or the development of proprietary interfaces to make different technologies compatible.

The modular approach of the invention also reduces the time required for system deployment. While combining the solutions of U.S. Pat. No. 8,493,223B2 and CN103513636B would require days or even weeks for planning, procurement and installation of fixed infrastructure (such as wiring for sensors, integration of communication modules, and compatibility settings), the present invention can be installed and set within a few hours. Compatibility with generic devices allows users to choose sensors and cameras that are widely available on the market, without the need for specific adaptations to meet proprietary protocols.

In terms of cost, combining the existing solutions would imply not only a high initial investment in fixed infrastructure (such as wiring and specific modules), but also recurring maintenance costs due to the need to maintain and replace proprietary or specialized equipment. The proposed invention, on the other hand, reduces these costs by using devices that are widely available on the market, without dependence on a specific manufacturer, and by allowing modular expansions as needed, without requiring a full system reset.

Thus, the present invention simplifies the implementation and operation process compared to the combination of patents U.S. Pat. No. 8,493,223B2 and CN103513636B, becoming a more practical, accessible, and scalable solution for monitoring activities in confined spaces.

Patent CN103513636B, in turn, presents a wireless monitoring solution with a convergence module, implementation elements, and a radio unit, integrated into a single box that is designed to monitor environmental factors such as oxygen, carbon monoxide, and hydrogen sulfide. Although it does not rely on a fixed infrastructure, this solution requires substantial adjustments to incorporate new sensors and integrate other systems.

In view of this document, the following stand out as significant distinguishing features of the system of the present invention: the simplified integration of different devices available on the market, such as cameras and wearable devices, without requiring unification of all modules in a single box. It is worth noting that, unlike the aforementioned document, which requires significant adjustments to the structure to support new sensors or technologies, the system presented herein is modular and allows for quick expansion. Furthermore, it was designed to operate based on different communication protocols, facilitating interoperability and adaptation to different operational scenarios, whereas CN103513636B is more dependent on a single communication configuration.

Considering the combination of U.S. Pat. No. 8,493,223B2 and CN103513636B, it is still possible to find the technical and functional originality of the system and method of the present invention, such as, for example, its modular and flexible architecture. This is because, unlike the rigid integration between hardware and proprietary sensors of the solutions given in U.S. Pat. No. 8,493,223B2 and CN103513636B, a modular architecture is adopted to facilitate the addition of new devices independently, which not only allows for an easier initial configuration, but also enables future expansions without the need for substantial system reconfiguration. Furthermore, while U.S. Pat. No. 8,493,223B2 and CN103513636B are dependent on proprietary communication devices and protocols, the present invention is agnostic with respect to communication protocols and devices, which allows it to operate with a variety of sensors and cameras widely available on the market, making integration more cost effective and faster. This level of flexibility is not evident in the combination of U.S. Pat. No. 8,493,223B2 and CN103513636B.

The computer-implemented system and method for integrated monitoring of hazardous activities in confined spaces, as described in the present invention, stand out by incorporating artificial intelligence for early anomaly detection and real-time response. This ability to continuously analyze collected data and generate immediate alerts is not covered by U.S. Pat. No. 8,493,223B2 and CN103513636B, making the integration of AI with generic devices, with the aim of improving the operational safety in environments that pose significant risks to workers, a unique innovation.

Furthermore, the solution presented can be distinguished with regard to the initial modular operation. While U.S. Pat. No. 8,493,223B2 requires the installation of wired gas sensors and cameras from the beginning, the System and Method of the present invention can start operating with cameras only. Furthermore, not all sensors have to be integrated at once, as in CN103513636B, allowing the system to be gradually adapted as needed, without impacting functionality.

For example, the System and Method of the present invention can initially be implemented using only standard IP cameras, allowing a basic real-time monitoring without the need for additional sensors or fixed wires or cables, as required in U.S. Pat. No. 8,493,223B2. This means that, in an urgent deployment scenario, such as a construction project underway or routine maintenance in a confined space, the team can set up the system in just a few hours, simply by installing the cameras and connecting them to the monitoring platform. In contrast, U.S. Pat. No. 8,493,223B2 requires a fixed wiring infrastructure to connect sensors and cameras to the monitoring module, which can take days to install and adjust. CN103513636B, in turn, requires the assembly of a single box with multiple integrated modules, which also demands considerable installation and adjustment time before it is fully operational.

Due to the use of widely available devices and the absence of proprietary components, the solution tends to have significantly lower acquisition and maintenance costs than the combination of solutions provided in U.S. Pat. No. 8,493,223B2 and CN103513636B, which require specific components and sensors. This feature not only reduces the total cost, but also facilitates the quick replacement of parts and maintenance of the system as a whole. Also, the dependence on a specific manufacturer increases the implementation cost, since one cannot opt for more affordable solutions or those that are readily available on the market.

Therefore, it can be stated that, although both solutions of U.S. Pat. No. 8,493,223B2 and CN103513636B deal with monitoring systems in confined spaces, the integration between them would require substantial modifications at both the hardware and software levels, making implementation more complex and costly. In the instance of U.S. Pat. No. 8,493,223B2, the system relies on the connection between the monitoring module and the external unit via an umbilical cable, which imposes physical restrictions and limits mobility. CN103513636B, while using wireless monitoring technology, requires the unification of multiple modules (environmental, location, and control modules) into a single box, which increases the complexity of both the assembly and operation. Integrating these two solutions would require a new in-depth study of communication protocols and sensor architecture, as well as alignment between different forms of data transmission (wired and wireless communication), resulting in a more complex, expensive, and difficult-to-maintain system.

Regarding the originality compared to the combination of U.S. Pat. No. 8,493,223B2 and CN103513636B, even considering the combination of the features of both, such an integration does not result in the same innovation presented by the System and Method of the present invention, since it would not offer the same flexibility and modularity; the ability to integrate different devices independently; the possibility of real-time analysis supported by AI for anomaly detection and response; and the ease of acquisition and cost-effectiveness associated with the use of generic devices.

Therefore, even if a skilled person would attempt to combine the solutions described in U.S. Pat. No. 8,493,223B2 and CN103513636B, they would not achieve the same proposed invention, as this one presents significant improvements in terms of simplified integration, greater flexibility, improved operational efficiency, and cost reduction. The modular architecture of the present invention eliminates the complexity associated with adapting distinct systems, allowing the incorporation of new devices without the need for extensive reconfiguration or reliance on fixed infrastructure. Furthermore, by being compatible with a wide variety of sensors and communication protocols, the system enables a more agile and cost-effective implementation, something that would not be possible by combining the abovementioned solutions.

U.S. Pat. No. 10,674,965B2, in turn, is described as a monitoring system based on specialized wearable devices that monitor the safety and productivity of workers in hazardous environments. The system uses proprietary algorithms to detect physical activity and calculate risk metrics, focusing on performance analysis and operational safety.

The solution provided in this document relies on proprietary wearable devices to capture physical activity data and calculate risk metrics, which limits the system's flexibility and interoperability. The system of the present invention, on the other hand, can integrate cameras, gas sensors, and generic wearable devices, without the need to rely on a single manufacturer. This allows the system to be adapted to different types of environments and monitoring needs, as well as reducing implementation and maintenance costs.

Furthermore, said document focuses on the analysis of post-operation productivity and performance; the system of the present invention is designed to detect anomalies in real time and issue instant alerts in case of hazardous scenarios, prioritizing the immediate safety of workers in confined spaces. Such capability that is supported by artificial intelligence is a significant advantage, as it allows for a quick response to hazardous conditions, such as workers falls, leaks of dangerous gases, or communication issues.

It is noteworthy that the aforementioned document requires a specific setting to connect wearable devices to the monitoring platform, which may limit the system's adaptation to new scenarios or the integration with other technologies, whereas the system of the present invention uses a modular architecture that facilitates the addition or replacement of devices without reconfiguring the entire system, increasing efficiency and scalability. Also, the aforementioned document relies on proprietary communication protocols for connecting wearable devices, which may limit compatibility with other sensors or technologies, whereas the system of the present invention is agnostic with respect to communication protocols, allowing interoperability with a wide range of devices and sensors on the market, ensuring greater adaptability and reducing integration barriers.

In addition to the recited particularities, which highlight the superiority of the System and Method of the present invention compared to the technology of patent U.S. Pat. No. 10,674,965B2, it is possible to emphasize the originality and efficiency of the new solution, even in view of the combination of U.S. Pat. No. 8,493,223B2, CN103513636B and U.S. Pat. No. 10,674,965B2. Such combination attempts to integrate environmental monitoring (U.S. Pat. No. 8,493,223B2 and CN103513636B) with wearable devices (U.S. Pat. No. 10,674,965B2) by providing a more complete view of the work environment and worker productivity. However, such an integration would not be trivial, as each of these patents was designed with distinct architectures, requiring significant adjustments for compatibility.

U.S. Pat. No. 8,493,223B2, for example, relies on umbilical cables for data transmission, which requires fixed physical infrastructure and restricts system mobility. To integrate it with CN103513636B, which uses a wireless module with integrated elements, the development of hybrid communication interfaces between wired and wireless transmission would be necessary, as well as adjustments to data synchronization. Such an adaptation would involve substantial modifications to both hardware and software, including new data conversion layers and adjustments to the transmission and reception of information between different types of sensors.

Also, U.S. Pat. No. 10,674,965B2 focuses exclusively on proprietary wearable devices designed to capture worker data in isolation. To combine this technology with environmental sensors and cameras, it would be necessary to redesign the data analysis model to correlate productivity metrics with real-time environmental information. This would require an in-depth reconfiguration of the software, creating a highly complex and difficult-to-maintain system.

In contrast, the present invention dramatically simplifies implementation by eliminating the need to make different technologies compatible with each other, allowing the system to operate natively with sensors, cameras, and wearable devices that are widely available on the market. While combining the three patents would require weeks for hardware, firmware, and communication protocol adjustments, the present solution can be installed and configured in just a few hours, simply by connecting compatible devices to the system.

The combination of U.S. Pat. No. 8,493,223B2, CN103513636B and U.S. Pat. No. 10,674,965b2 does not offer the same real-time monitoring capability as the computer-implemented system and method for integrated monitoring of hazardous activities in confined spaces of the present invention. Document U.S. Pat. No. 10,674,965B2, for example, is more focused on post-operation analysis, while U.S. Pat. No. 8,493,223B2 and CN103513636B deal with environmental and communication data in confined spaces, without a direct integration with artificial intelligence to issue instant alerts. The computer-implemented system and method for integrated monitoring of hazardous activities in confined spaces, in turn, provides early hazard detection and immediate response, which is essential in hazardous environments such as confined spaces. The combination of U.S. Pat. No. 8,493,223B2, CN103513636B, and U.S. Pat. No. 10,674,965B2 does not provide this capability in an integrated and efficient manner.

Furthermore, combining the three solutions would require significant infrastructure investments, as it would demand proprietary equipment and sensors, as well as adjustments to the communication between devices operating on wired and wireless networks. The proposed invention, by adopting a modular and open architecture, significantly reduces operational and maintenance costs, allowing for the replacement or addition of new devices without the need for system reconfiguration.

Therefore, even considering the possible combination of U.S. Pat. No. 8,493,223B2, CN103513636B and U.S. Pat. No. 10,674,965B2, the System and Method of the present invention remains a superior solution, as it offers a simpler implementation system, more efficient operation and lower cost, eliminating the technological and structural barriers of the existing solutions.

Document U.S. Pat. No. 11,954,751B2 is also part of the general state of the art and focuses on safety monitoring of work carried out on scaffolds, ladders, and elevated platforms. The system is designed to ensure the safe use of such equipment through integrated sensors and short-range communication, generating alerts and safety scores based on the users'behavior.

While the described solution focuses on the safe use of equipment by monitoring the users'behavior during the operation of specific tools, such as ladders and scaffolds, the system of the present invention is designed to directly monitor workers in confined spaces, focusing on real-time personal safety, including the detection of anomalies such as falls, lack of movement, or the presence of hazardous gases. This makes it more comprehensive and targeted at monitoring risks to humans, not only the use of equipment.

Another issue to be highlighted is the integration of generic devices. Said document relies on sensors integrated into the equipment to capture usage data, whereas the system of the present invention uses generic video devices, such as cameras and gas sensors, which may be easily replaced or added as needed, hence increasing flexibility and reducing the acquisition costs. Furthermore, a modular architecture was adopted to allows for the expansion and integration of new devices without the need to reconfigure the basic system structure, which is not seen in the aforementioned document, which has a more limited scope, since integration is focused on monitoring specific equipment.

It should be noted that, although the aforementioned document has the functionality to issue alerts based on events of unsafe use of equipment, it does not provide a continuous analysis of working conditions in real time, as the system of the present invention does. The detection of immediate risks, supported by artificial intelligence, is a clear advantage of the present solution.

When simulating the combination between the four patents, it can be stated that it would result in an extremely complex system, with different proprietary devices and protocols for monitoring environmental factors, safety wearables, and equipment. This would require deep hardware and software integration, as well as alignment between different forms of communication (wired, wireless, and short-range communications). The combination would not offer a unified focus on real-time safety monitoring of the worker, as each patent has distinct objectives: U.S. Pat. No. 8,493,223B2 and CN103513636B focus on environmental factors, U.S. Pat. No. 10,674,965B2 on productivity and safety through wearables, and U.S. Pat. No. 11,954,751B2 on the safe use of equipment. The System and Method of the present invention, by integrating different types of devices (cameras, gas sensors and wearables) in an agile and efficient manner, provides a comprehensive view of working conditions in real time, something that the combination of the four patents would not be able to achieve, even after complex reconfigurations.

Importantly, implementing a combination of all four solutions tends to result in a high-cost system due to the need for proprietary components and specific sensors. Furthermore, maintenance would be more complex, as it would require expertise in different technologies and communication protocols, whereas the System and Method of the present invention makes use of devices widely available on the market, reducing both the initial acquisition cost and the maintenance cost over time. Furthermore, its faster and less complex implementation, for not requiring specific integration of different proprietary systems, makes this technology unique and inventive.

Another analysis performed was the combination of U.S. Pat. Nos. 8,493,223B2, 10,674,965B2 and 11,954,751B2, which would focus on combining environmental safety monitoring (U.S. Pat. No. 8,493,223B2), wearable safety devices (U.S. Pat. No. 10,674,965B2) and specific safety equipment (U.S. Pat. No. 11,954,751B2). As previously highlighted, integrating these three solutions would create a more complex monitoring system, requiring hardware and communication adjustments at each stage of the process.

The computer-implemented system and method for integrated monitoring of hazardous activities in confined spaces is capable of continuously monitoring workers and the environment, identifying anomalies and issuing real-time alerts, something that would be more difficult to achieve with the integration of the three solutions, due to differences in technologies and communication protocols involved. Emphasis can also be placed on the adaptability of the solution to be patented, because while the combination of U.S. Pat. Nos. 8,493,223B2, 10,674,965B2 and 11,954,751B2 would result in a more rigid and expensive system, the System and Method of the present invention offers a more flexible, adaptable and low-cost solution that can be tailored according to the needs for monitoring in confined spaces.

A potential integration between U.S. Pat. No. 8,493,223B2, which is focused on monitoring environmental factors in confined spaces using fixed cables and dedicated sensors, and U.S. Pat. No. 10,674,965B2, which is focused on wearable devices for monitoring worker productivity and safety, would add wearables to the environmental monitoring system, which would require substantial adjustments to communication, wiring infrastructure, and connectivity between different types of sensors (environmental and wearable sensors).

The System and Method of the present invention is already designed to integrate wearable devices and environmental sensors in a modular way, without the need to adjust the physical infrastructure or reconfigure the communication system. Furthermore, while U.S. Pat. Nos. 8,493,223B2 and 10,674,965B2 would have communication and latency limitations due to the complex infrastructure and the use of different protocols, the System and Method of the present invention can detect anomalies in real time, by integrating data from wearables and environmental sensors into a single platform, without the need for significant resetting, making it unique.

Integration of patent CN103513636B, that is designed for wireless environmental monitoring dependent on a central convergence module, which limits its adaptability, and U.S. Pat. No. 11,954,751B2, which focuses on safety monitoring of specific equipment such as ladders and scaffolds using short-range sensors to generate safety scores, would require adjustments to the wireless infrastructure, as well as combining short-and long-range communication protocols, making the system more expensive, more complex, and more difficult to maintain.

The computer-implemented system and method for integrated monitoring of hazardous activities in confined spaces, being a communication protocol-agnostic solution, facilitates the integration of environmental and wearable sensors without the need to adjust the system's structure. Additionally, unlike U.S. Pat. No. 11,954,751B2, which prioritizes equipment, the System and Method of the present invention monitors directly the workers by detecting risks to personal safety, such as adverse environmental conditions and incorrect use of equipment, in real time.

In light of the foregoing, it is clear that the present invention provides improvements over the prior art for being independent of a fixed infrastructure, since it allows for an initial setting only with cameras, without the immediate need for additional sensors or fixed cables, ensuring greater mobility, ease of implementation, scalability, and adaptation to operational needs.

Unlike the patents recited above, which rely on proprietary devices and protocols, the proposed solution is manufacturer-agnostic and compatible with a wide range of sensors and devices on the market. This reduces costs and implementation time, while also providing a simplified integration. The reliance on proprietary components of the recited patents increases the cost for implementing and maintaining the existing solutions, whereas the proposed invention minimizes these costs by using devices that are widely available on the market, allowing for replacement and upgrades without the need for a full system reset.

Furthermore, it stands out for being easily adaptable to add gas sensors or other devices as needed, without modifying the basic structure of the installation. While the cited patents would require substantial hardware and software adjustments to make different technologies compatible (such as the wired communication of U.S. Pat. No. 8,493,223B2 and the unified module of CN103513636B), the present invention adopts a modular and independent approach, eliminating technical barriers and significantly reducing implementation time and costs.

The system also offers a real time, continuous analysis of working conditions and can be designed to operate with different communication protocols, facilitating interoperability and adaptation to different operational scenarios, without depending on a single communication configuration. Interoperability with different devices and protocols facilitates the communication between workers and security teams, increasing the efficiency of operations and allowing for a better response to emergencies.

One extremely important factor is that, unlike existing solutions that focus on post-event analysis or segmented monitoring, the present invention incorporates artificial intelligence for anomaly detection and generation of instant alerts, allowing for an immediate response in hazardous situations.

It is also important to highlight that the present invention offers advantages including improvements in safety, operational efficiency, and regulatory compliance. With continuous, real-time monitoring of hazardous work sites, such as confined spaces and work at height, it is possible to prevent accidents and incidents.

Furthermore, early detection of anomalies, such as leaks of hazardous substances or equipment failures, allows for rapid intervention to prevent damage or injury. The integration of various communication devices facilitates a more effective communication between workers and those responsible for safety, improving the coordination of operations and response to emergencies.

Technology also contributes to compliance with safety and environmental regulations and enables adaptation to different work sites, including adverse conditions. Early detection of anomalies, continuous monitoring capabilities, and the flexibility to adapt to different environments ensure greater compliance with occupational safety standards and environmental regulations, contributing to a safer and more efficient work environment.

### BRIEF DESCRIPTION OF THE INVENTION

The present invention relates to a computer-implemented system and method for integrated monitoring of hazardous activities in confined spaces, which allows real-time monitoring using commercially available devices for data collection. This includes the use of environmental sensing, cameras, and wearable devices and enables real-time monitoring both locally and remotely. Furthermore, the system is capable of issuing autonomous alerts and detailed reports that indicate non-conformities and risky situations, and correlates variables to generate valuable data, predict accidents, and increase safety. The system further manages and stores all collected data in the cloud, allowing for future access for audits and forensic investigations. Thus, the proposed computer-implemented system and method offer solutions that address and minimize the difficulties encountered in monitoring confined spaces. Furthermore, the present invention encompasses a non-transient, computer-readable storage medium that stores instructions which, when processed by a computer, enable it to execute the steps of the integrated method of monitoring hazardous activities in confined spaces, as defined above.

### BRIEF DESCRIPTION OF THE FIGURES

To provide a comprehensive and full view of the goal of the present invention, the figures are presented below, which are referred to as follows.

FIG. 1 schematically represents an overview of the monitoring system architecture.

FIG. 2 schematically shows the services and network topology of the monitoring system.

FIG. 3 schematically represents the services and components of the sensor data acquisition and supply flow.

FIG. 4 schematically represents the communication architecture between the monitoring system and the gas sensor devices.

FIG. 5 schematically represents the user management and authentication services.

FIG. 6 schematically represents the video monitoring services and components.

FIG. 7 schematically depicts the video segment persistence flow for later monitoring.

FIG. 8 schematically represents the services and components of the sensor data acquisition flow.

FIG. 9 schematically represents the data acquisition and storage flow from the sensors.

FIG. 10 schematically represents the life cycle of an operation in the monitoring system.

FIG. 11 schematically represents the data streaming communication model.

FIG. 12 schematically represents the workflow for live video monitoring using a video management system.

FIG. 13 schematically represents the execution flow of video segment persistence for later monitoring.

FIG. 14 schematically represents the synchronization services between instances of the computer-implemented method and the cloud.

FIG. 15 schematically represents the navigation menu of the web platform.

FIG. 16 shows a schematic representation of the login screen.

FIG. 17 schematically represents the password reset screen.

FIG. 18 schematically represents the user management screen.

FIG. 19 schematically represents the user registration and editing screen.

FIG. 20 schematically represents the user permission editing screen.

FIG. 21 schematically represents an overview of the dialog screen (pop-up) describing user roles and permissions on the web platform.

FIG. 22 schematically represents the overview screen of the list of registered devices.

FIG. 23 schematically represents the (pop-up) screen for editing and registering new sensor devices.

FIG. 24 schematically represents the dialog box (pop-up) for editing and registering image devices (cameras).

FIG. 25 schematically represents an overview of the operations list.

FIG. 26 schematically represents the monitoring dashboard for the “Live View” tab of an operation with pending status.

FIG. 27 schematically represents the monitoring dashboard for the “Live View” tab of an operation with an ongoing status.

FIG. 28 schematically represents the operation data tab.

FIG. 29 represents schematically the deviations and alerts tab.

FIG. 30 schematically represents the geographic data tab.

FIG. 31 schematically represents the monitoring dashboard of recorded operations.

FIG. 32 schematically represents the operation of the system, highlighting the sensors used inside the confined space to monitor the personnel performing the services. On the outside the attendant monitors the work being carried out inside the confined space; it's possible to remotely monitor the operation by someone not physically present on site, provided there is internet access for live streaming.

FIG. 33 schematically represents the activity flow of the system, being organized in steps.

### DETAILED DESCRIPTION OF THE INVENTION

The present invention relates to a computer-implemented system and method for integrated monitoring of hazardous activities in confined spaces, which allows real-time monitoring using commercially available devices for data collection. This includes the use of environmental sensing, cameras, and wearable devices, in addition to enabling real-time monitoring both locally and remotely, providing a portable solution that allows for real-time tracking of activities in the cloud.

The solution developed aims to facilitate the supervision of workers' activities in confined spaces, providing the operation attendants with a real-time monitoring tool that assists in quick decision-making when an incident is identified. Furthermore, it seeks to record and store detailed data for later analysis and incident investigation, aiming to identify patterns, trends, and areas for improvement in security and operations management.

To that end, a hand-held solution was developed in which a computer-implemented method or system (set of instructions developed in the method) was elaborated, installed, configured, and run locally to ensure the safety and well-being of workers operating in environments with restricted access, such as confined spaces.

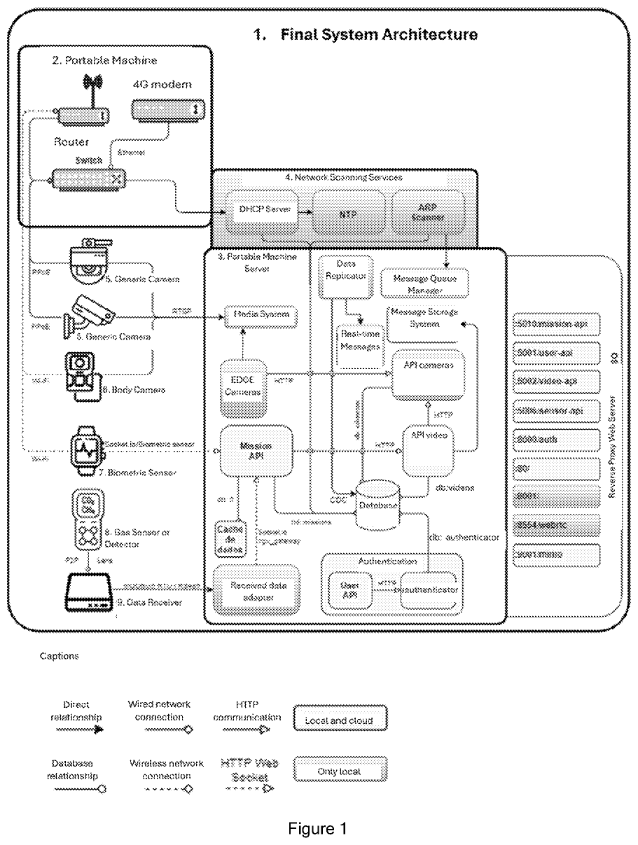

The computer-implemented method for integrated monitoring of hazardous activities in confined spaces, represented in FIG. 1, comprises the following steps: 1) Using a portable machine (2) with a server (3); 2) Taking the portable machine to a location where a hazardous activity is performed in confined spaces; 3) Installing the devices in the confined space and on the worker; 4) Performing the activity in the confined space by the worker; 5) Collecting real-time data on the progress of the activity with the following devices: generic cameras (5), body cameras (6), and O₂, H₂S, CO and CH₄ sensor data (7); 6) Sending the data for monitoring; and 7) Presenting the data to the users.

After viability studies, the proposed system infrastructure includes two independent parts: an online information center services and a portable machine deployed to the space where the missions/operations are carried out. A portable device, for example, could be a laptop encased in a briefcase or another device (tablet, cell phone).

The specifications for the portable machine must meet the following criteria:
(1) have at least two network interfaces, one ethernet interface for the local network, connected to the local address switch, to be used with devices and with addressing managed by the local Dynamic Host Configuration Protocol (DHCP) service, and another interface for external connectivity, represented by 4G/5G connectivity;
(2) managing services through container isolation (using Docker and Docker Compose initially);
(3) have a universal serial bus (USB) RS-485 modbus converter connected to the Data Receiver together with the Ventis Pro 5 portable gas sensor.

The monitoring system's hardware infrastructure consists of a Linux server, but other servers can be used, such as Windows, acting as the central core, hosting microservices in Docker containers to facilitate scalability and efficient management of resources. Docker is an open-source platform, and a Docker container is an isolated environment that contains all the parts of a computer system necessary to run an application. Unlike virtual machines, Docker containers are lighter and faster because they share the host operating system, allowing multiple applications to run in different environments.

The monitoring system of workers within confined spaces is an on-premise solution, meaning that the computer-implemented method is installed, configured, and run locally on the servers and infrastructure of a company or organization. The system uses a microservice-based architecture to provide flexible and scalable functionality.

FIG. 1 shows the final system architecture (1), with the interactions between the items, devices and services of the local monitoring system. It is important to note that the direction of the connecting arrows indicates which item (origin of the arrow) depends on another (destination of the arrow). The main constituent elements of the system are: (2) portable machine, (3) portable machine server, (4) network scanning service, (5) fixed camera, (6) body camera, (7) biometric sensor, such as a smart watch, (8) gas sensors or detectors, connected to a data receiver.

The system is composed of several microservices, each being responsible for a specific and independent function and developed and deployed separately, which allows for autonomous updates and maintenance. This promotes horizontal scalability, where each microservice can be individually sized as needed. Such modularity, scalability, and operational efficiency of the monitoring system allow for easy future adaptations and expansions.

Microservices communicate with each other through communication protocols such as hypertext transfer protocol/representational state transfer (HTTP/REST), real-time communication channels such as WebSocket, or asynchronous messaging. This communication, which can occur synchronously or asynchronously depending on the requirements of each service, provides access to the same instance of the DBMS database for data storage and retrieval, ensuring information consistency and integrity throughout the system and allowing services to share information efficiently.

The relational database management system used is open-sourced, reliable, robust, and capable of handling large volumes of data efficiently. Another feature is its scalability and compatibility with a wide variety of platforms and operating systems. Therefore, the reliability and efficiency of the system are guaranteed.

The monitoring system hardware infrastructure initially consists of a Linux server, acting as the central core, hosting microservices in Docker containers, which technology allows to pack, distribute, and run applications in isolation within a lightweight and efficient environment, facilitating scalability and resource management. With the Docker, microservices can be run he same way in any environment (whether on a portable server, a cloud server, or another system). This avoids compatibility issues between different operating systems or configurations.

Docker containers include all the dependencies necessary for microservices to function, such as libraries, frameworks, and specific tools. This eliminates the need to install dependencies directly on the host operating system. Furthermore, they allow each microservice to be scaled separately, with new containers being created or terminated as needed by the system.

The system uses Docker and Docker Compose to manage and orchestrate microservices on the portable server. Docker Compose is used to define and run multi-container applications, making it easier to manage interdependent services. Since the portable machine supports container isolation using Docker, all microservices can run independently on a single portable hardware device.

During an operation, the portable server, upon activation, automatically starts the set of Docker containers, each responsible for performing a specific function in the integrated monitoring system. The containers responsible for acquiring sensor data, transmitting video, and authenticating users start operating simultaneously, ensuring that the system is fully functional from the start of execution.

Among the containers, the Cameras application programming interface (API) can be mentioned, which establishes a connection with the cameras configured in the environment, capturing and transmitting images in real time. These streams are then forwarded to the Video API container, which stores the video segments in the Min.io storage service. This process allows for both live monitoring and later access to recordings.

Interaction between containers occurs through standardized communication protocols, such as HTTP/REST and WebSocket, promoting an efficient and real-time exchange of information. Such a modular and connected architecture ensures that all functionalities operate harmoniously, allowing for continuous and reliable integration of the described services.

Regarding the network, a switch was connected to the server to provide Ethernet connectivity for local devices, while a router connected to the switch and a 4G modem provided wireless connectivity and internet access, enabling remote operations. A DHCP server microservice, a Python-implemented protocol that allows for the automatic assignment of network parameters to devices, was responsible for defining IP (Internet Protocol) addresses, base on a database mapping table, hence ensuring a reliable connectivity and efficient network management. Also, a NTP (Network Time Protocol) service was used to synchronize the clock of all devices, ensuring a consistent time reference for precise and coordinated monitoring operations.

The monitoring system further includes a network scanning service based on the ARP (Address Resolution Protocol) protocol, i.e., ARP Scanner. This tool identifies and maps IP addresses to MAC (Media Access Control) addresses, allowing the identification of active devices on the local network. Such detected network events were published on the RabbitMQ messaging service, an open-source server developed using the Erlang programming language, which is scalable and flexible, allowing applications to communicate efficiently and securely.

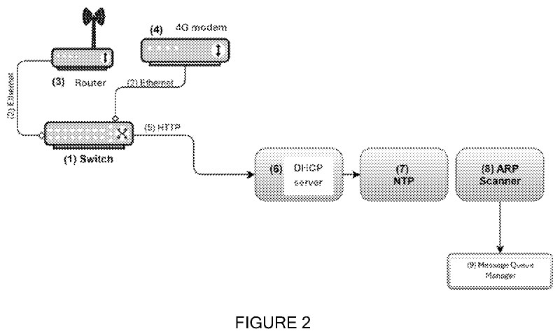

FIG. 2 schematically presents the services and network topology of the monitoring system, with the architecture composed of hardware and software elements that work together to manage communication and connected devices. A network switch (1) connected to the server provides Ethernet connectivity (2) to local devices, ensuring stability and high bandwidth, forming the basis of connectivity for the devices, while a router (3) connected to the switch and a 4G modem (4) provide wireless connectivity and internet access, enabling remote operations.

The switch connects via HTTP communication (5) to a Python-implemented, DHCP (Dynamic Host Configuration Protocol) microservice (6) that is responsible for dynamically assigning IP addresses to devices connected to the network based on a mapping table in the database, eliminating the need for manual settings to be entered for each device and ensuring a reliable connectivity and efficient network management. In parallel, a NTP (Network Time Protocol) server (7) synchronizes the clocks of all devices on the network, ensuring that event records and logs have consistent timestamps, which is essential in distributed systems.

Another important component is the ARP (Address Resolution Protocol) Scanner (8), which monitors the local network for connected devices, identifying their MAC (Media Access Control) addresses. This functionality provides an efficient control and detection of active devices on the network, which, when detected, are published in the message queue manager (9), which plays the middleware role for asynchronous communication between the various components. It facilitates the exchange of messages between the ARP Scanner and other services, ensuring that data travels reliably and in a scalable manner.

This architecture is efficient in real-time monitoring scenarios, such as sensor networks. Integration between hardware elements, such as the switch and router, and system components, such as DHCP, NTP, and message queue manager, ensures a stable communication, efficient device management, and a continuous and synchronized data flow.

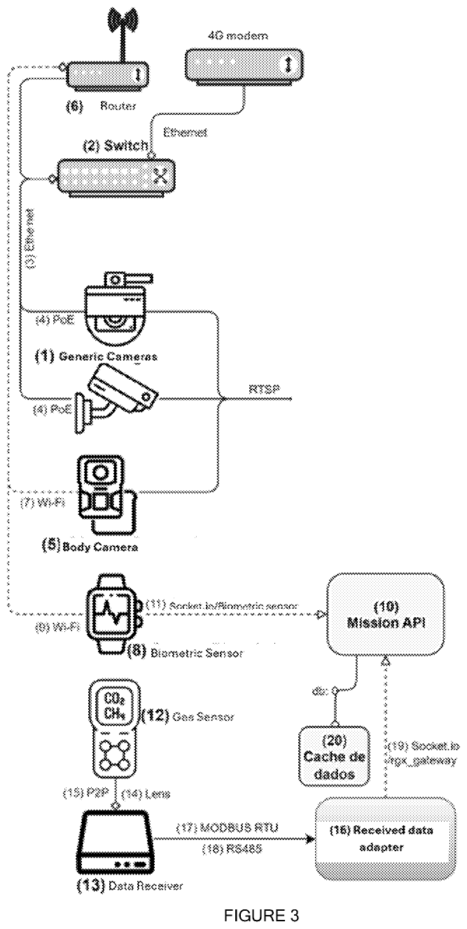

Services and components of the sensor data acquisition and supply flow are presented in FIG. 3 through the integrated architecture, which connects the physical devices and the system for an efficient real-time monitoring of activities in challenging environments, such as confined spaces. To capture real-time images, two generic fixed cameras (1) connected to the switch (2) via Ethernet cables (3), using the PPPoE (Point-to-Point Protocol over Ethernet) protocol (4) were used, as well as a body camera (5) that acts as a wearable device offering mobility and real-time recording, and for being a wireless device, was configured to make a direct connection to the router (6) via Wi-Fi (7). Both types of cameras provided RTSP (Real Time Streaming Protocol) streaming for video transmission.

In addition to the cameras, a biometric smart watch-type sensor (8), provided with the Wear OS operating system, was used to capture biometric data from workers, such as heart rate, and geolocation, being of importance for real time monitoring the health and location of workers. These devices are connected via a dedicated wireless interface (9), ensuring the constant transmission of biometric and geographic data. A specific application on the smartwatch sent the collected information to the Mission API (10) via a WebSocket communication protocol (11), which maintains an open connection, allowing continuous communication with the monitoring system.

To integrate IoT sensors, such as the gas sensor (12), into the monitoring system, an intermediate data receiver (13) was required due to the communication protocol of the device, which sends measurements via Leans (14), which is an open source protocol using P2P (Peer to Peer) technology (15) to allow direct communication between network nodes. This, in turn, connects to the server via the Received Data Adapter (16) using the Modbus RTU (Remote Terminal Unit) protocol (17) over the RS-485 physical layer (18), used as a data transmission method that allows the exchange of information using single cable between transmitters and receivers.

Such configuration allowed the gas measurements (O₂, H₂S, CO, and CH₄) to be transmitted efficiently and recorded on the server. The Modbus RTU protocol, which is common in industrial environments, ensures a stable connection between the data receiver and the server, enabling integrated analysis of environmental data.

The data adapter forwards the data to the Mission API (10) also via the WebSocket communication protocol (19). This is ideal for transmitting this data in real time as it has low latency and automatically manages reconnections and transmission failures, ensuring that the data is delivered even under unstable network conditions. To ensure data efficiency and availability, the system includes a data cache (20), which acts as an intermediate layer to store temporary information before sending it to the database.

This architecture is designed to ensure a continuous and reliable flow of information. Communication between physical devices and systems occurs through standardized protocols, allowing data to be analyzed in real time to ensure security and operational efficiency. The modular and integrated design of the solution makes it ideal for critical environments where precision and reliability are essential.

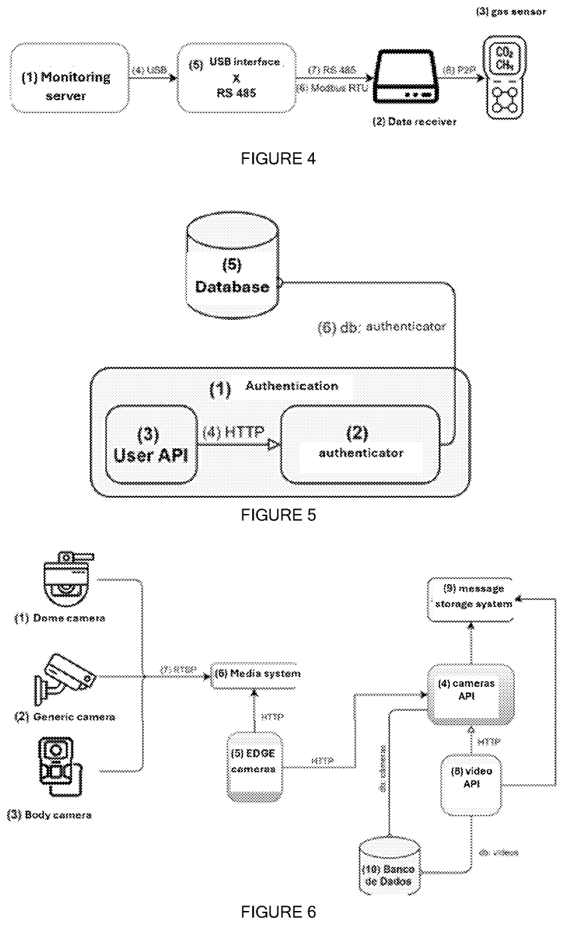

FIG. 4 shows schematically the architecture of the communication flow between the monitoring server (1), the data receiver (2) and the gas detection sensor (3). The system uses a series of protocols and interfaces to ensure connectivity between components.

The monitoring server centralizes data acquisition and analysis and is connected, via USB (4), to a USB to RS-485 interface (5), allowing communication between the computer with a USB port and devices using the RS-485 communication protocol. This adapter converts digital signals sent through the USB port into signals suitable for the RS-485 bus and vice versa, enabling integration between technologies. This protocol is widely used in industrial applications due to its robustness and ability to operate over long distances.

After conversion, the data is transmitted via the Modbus RTU protocol (6) through an RS-485 connection (7) to the data receiving device (2). This device acts as a gateway, mediating communication between the server and the sensors, interpreting commands received via Modbus RTU and converting them into a protocol compatible with the sensors connected to it.

Finally, the gas sensor (2) communicates directly with the data receiver using a specific serial protocol, in this case, P2P (8). This protocol facilitates the exchange of information, allowing gas concentration data to be collected and sent back to the monitoring server for real-time analysis.

This architecture ensures an efficient integration between monitoring systems and industrial sensors, even in challenging environments. The use of standardized conversion interfaces and protocols provides a reliable and compatible communication between the different system components.

### Microservices

The architecture of the monitoring system of confined spaces was based on microservices, which offers flexibility, scalability, and modularity to the system as a whole. The system was composed of several microservices, each playing a specific role, such as mission management, user authentication, video processing, and sensor data acquisition.

The platform's main services are:
(*) Mission API: responsible for managing operations in confined spaces, device management, and data collection from devices. It uses a high-performance, open-source cache service for in-memory data storage, to optimize operation performance and efficiency.
(*) User API: manages user authentication and authorization, integrating with an open-source authentication server for identity and access management.
(*) Cameras API: identifies and manages cameras
connected to the monitoring system local network. Creates HLS (HTTP Live Streaming) live streams from RTSP (Real Time Streaming Protocol) streams from online cameras.
(*) Video API: consumes the HLS streams created by the Cameras API and saves the video segments to a high-performance, open-source message storage server for future playback and access to the recordings.
(*) Cameras EDGE: publishes RTSP streams from cameras registered in the database by the Cameras API on a WebRTC (Web Real-Time Communication) channel, an open-source technology that enables real-time communication through an instance of the media service.
(*) Data adapter: connects to the gas sensor data receiver to periodically collect data and send it via WebSocket protocol to the Mission API.

A User API is a microservice responsible for facilitating interaction between applications and the authentication server, which in turn is a robust identity and access management platform. Its primary role is to oversee critical aspects such as user authentication, permission control, and user profile-related data storage.

By establishing direct communication with the authentication server, the User API simplifies the complexity involved in managing permissions and authentication for the applications that use it. In practical terms, this means that applications don't need to interact directly with the authenticator; instead, they can make requests to the User API, which then handles all the necessary operations.

Therefore, the User API acts as an abstraction layer, providing a more user-friendly and cohesive interface for applications to communicate with the authenticator. This approach was adopted with the aim of simplifying integration, reducing complexity and coupling between applications and the identity management server. Furthermore, it provides greater flexibility and extensibility, allowing the business logic related to authentication and permissions to be customized and extended as needed, without directly affecting integration with the authenticator.

In the authentication process, there is a direct interaction with the authenticator, where a client or user submits the necessary credentials. These credentials may include information such as username and password. The authenticator in turn checks these credentials and, if valid, generated a temporary access token. This token is then returned to the client, allowing access to protected resources within the system for a specified period. The token-based approach provides an effective means of ensuring user authenticity and authorization, while maintaining data security and integrity.

FIG. 5 shows the architecture of the authentication system used to manage secure user access. At the core of this process is a robust identity management and access control platform (1) that facilitates user authentication (2) and permission management (3).

Communication between the Authenticator (2) and the User API (3), developed in Python, is performed via HTTP requests (4). The User API serves as an intermediate between the main system and the authentication service, sending authentication requests, verifying credentials, and managing sessions to the authenticator. This workflow ensures that only authenticated and authorized users can access the system functionalities.

The database (5) is used to store user-related information, such as credentials, permissions and access settings. It is connected directly to the authenticator (6), which queries and manages this information efficiently during the authentication process.

Such modular and centralized structure offers several benefits, such as separating responsibilities between the user API and the authentication service, which increases the system security and scalability. Furthermore, using the authenticator as the core of identity management allows for integration with different authentication providers and the application of advanced access control policies, such as multi-factor authentication (MFA) and group and role management. This makes the system more flexible and prepared to meet different authentication scenarios in corporate and industrial environments.

Furthermore, system users are granted permissions according to their role:
(*) Supervisor: person authorized to create users, adjust permissions, reset passwords and manage operations. Its responsibilities include creating operations, defining profiles for operators and attendants, assembling the team for each operation, and comprehensively monitoring operational activities.
(*) Attendant: user authorized to approve operator check-ins and issue/receive alerts, being responsible for the total monitoring of operations.
(*) Emergency responder: responsible for monitoring operations and receiving alerts, providing support and assistance in case of accidents.
(*) Authorized worker: individuals in charge of performing the service and subject to monitoring during the activities. This user may request help via the panic button and access the data generated by the monitoring system.

Permissions were assigned to each type of user in the system, detailing what each one can do in terms of interaction with the data. This division of permissions is designed to ensure that each user has appropriate access to their responsibilities, promoting security and efficiency in system management.

The supervisor has broad and comprehensive permissions, including the following roles: viewing operations, ending operations, creating operations, initiating operations, and accessing to the panic button for emergencies. Furthermore, the supervisor can manage users and devices, allowing for complete control over the system and its resources.

The attendant has intermediate permissions. He can view operations, initiate operations, and use the panic button in case of emergency. However, he does not have access to create, terminate operations, or manage users and devices, his roles being restricted to monitoring and support.

The authorized worker and the emergency responder have the most restricted level of access. Their permissions are limited to viewing operations, with access only to the information essential to their roles, without the ability to change, create, or manage system elements.

Regarding integration of devices with the system, to ensure they were available on the platform to be monitored, registration on the Device Management screen was required. For each device, the fields: Name, Brand, Model, MAC Address, and Device Type have been defined as required fields. The types of supported devices were: Generic camera, Body camera, pan-tilt-zoom (PTZ) camera, Smartwatch, and Gas sensor.

For camera-type devices and their variants (such as body cameras and PTZ Camera), additional fields related to the RTSP stream settings were created. These fields included: Protocol, Username, Password, Port and RTSP stream Path.

When a valid device is registered, a unique fixed IP address is automatically assigned, which remains unchanged throughout the device's interaction with the monitoring system. To ensure the correct IP address assignment, the system used the MAC address provided during registration of the device.

In addition, it was defined that when a device attempts to connect to the monitoring system network, the corresponding MAC address is processed by a DHCP (Dynamic Host Configuration Protocol) server. Such DHCP server verifies if the MAC address is valid and if it is correctly registered in the system.

If all conditions are met, DHCP assigns the previously determined static IP address to the device, as registered in the device table. This approach ensures a consistent and reliable assignment of IP addresses to each device, facilitating its identification and management within the monitoring system.

The ability to view video in real time has been implemented in the system. FIG. 6 presents the detailed architecture for capturing, processing, analyzing, and storing videos in real time, with data from various sources, such as surveillance cameras and wearable devices.

In the initial part of the flow, three types of capture devices are found: a dome camera (1) that is widely used for monitoring large areas; a generic camera (2) that plays the role of conventional surveillance; and a body camera (3) that is designed for recording while in motion. First, the Cameras API service (4) is responsible for periodically updating the online or offline status of the cameras in the database.

Based on this information, the EDGE Cameras microservice (5) takes on the role of registering the stream in the Media System (6), an intermediary server that plays an essential role in managing and retransmitting these streams. All cameras transmit their video streams using the RTSP (Real-Time Streaming Protocol) protocol (7), which is widely accepted for real-time transmissions. The media system is then responsible for creating a video stream using the WebRTC protocol, allowing access to the live video in any browser. There was a need to retransmit the stream using WebRTC because the RTSP protocol is not natively supported by web browsers. Since the platform was developed as a web-based system, this retransmission is essential to ensure that the data can be efficiently consumed by subsequent components of the architecture.

Meanwhile, the Cameras API microservice was designed to create its own HLS stream (HTTP Live Streaming), a protocol that divides the video into small segments and delivers them to viewers in a sequence of playlists. This approach allowed for a more stable transmission that was adaptable to the viewer's bandwidth.

The Video API microservice (8) consumes the playlists created by the Cameras API, processing and saving the video segments in the Message Storage System (9). This system ensures that videos are stored securely, scalably, and accessibly, allowing users to retrieve and play back recordings as required.

Moreover, the EDGE Cameras (5) plays a critical role in the initial processing of videos, applying preliminary analyses, such as motion detection or anomalous patterns. After this processing, the data is sent to the Cameras API (4), which manages the integration with the storage system and prepares the streams for further analysis.

The Video API (8) service performs advanced analytics, such as extracting relevant information, detecting events, and preparing videos for reporting or playback. To support these operations, the system uses a Relational Database (10), where metadata associated with the videos, such as timestamps and detected events, are stored, ensuring a well-organized and easily accessible structure for queries.

This architecture was designed to maximize the efficiency and reliability of the monitoring system, using a modular and scalable approach. Integration of multiple components ensures compatibility, optimizes data processing, and guarantees its secure storage for real-time or retrospective analysis. The solution stands out for its robustness and ability to meet the demands of environments that require continuous monitoring and intensive video processing.

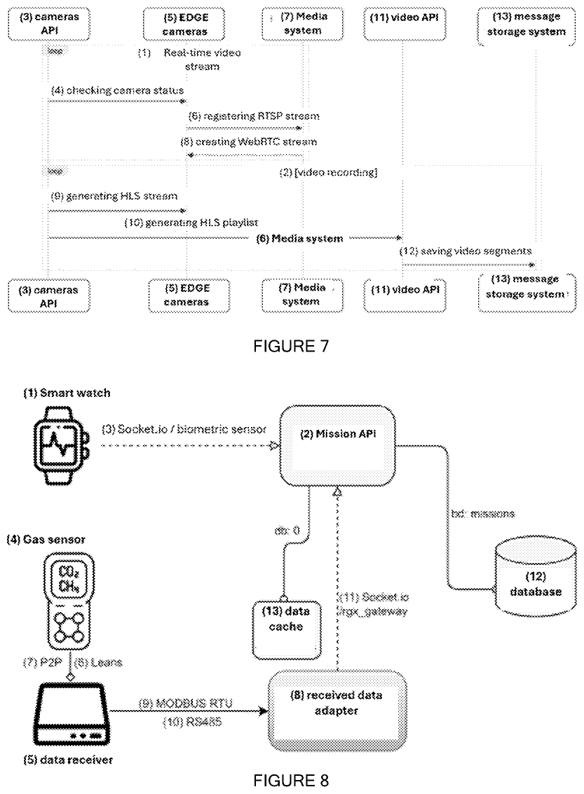

FIG. 7 illustrates a sequence diagram detailing the flow of operations related to video processing and management in the monitoring system, involving interconnected microservices. The processes are divided into two main contexts: real-time video streaming (1) and video recording (2), showing how the different components interact to perform these tasks.

The real-time video streaming process starts with the Cameras API (3), which continuously checks the status of connected cameras to determine whether they are online or offline (4). Once a camera is identified as active, the Cameras EDGE (5) records the video stream in RTSP protocol, sending it to the media system (6). The Media System (7) converts the RTSP stream into a browser-compatible format, creating a WebRTC stream (8). This protocol allows the real-time video to be accessed directly by browsers, overcoming the RTSP limitations, which is not natively supported by web applications. Communication between the components is presented in a continuous loop, ensuring that the system is always up-to-date with the active streams.

In parallel with streaming, the system also performs video recording (2). In this context, the Cameras API (3) creates a video stream in HLS (HTTP Live Streaming) format (9), which divides the video into small segments and organizes them into a playlist that is created by the Cameras API itself (10). This approach is ideal for adaptive streaming, allowing the video quality to be adapted to the bandwidth available.

Once the HLS playlist is created, it is consumed by the Video API (11), which collects the video segments (12) and stores them in the message storage system (13). This scalable and secure storage allows recorded videos to be available for later access and playback by users. The recording process also operates in a continuous loop, ensuring that all videos are recorded consistently.

The Cameras API plays a key role in continuously checking the status of the connected cameras, determining whether they are online or offline. It is also responsible for managing HLS (HTTP Live Streaming) video streams, organizing both real-time streaming and recording operations, and ensuring integration with other system components.

Cameras EDGE acts as an intermediary in processing video streams, recording the streams in RTSP protocol and forwarding them to the Media System. This component is essential for transforming raw camera data into a format that can be consumed by other services.

In turn, the Media System plays the role of retransmitting the video streams received from the Cameras EDGE, converting them to the WebRTC protocol. This retransmission is essential to enabling access to the video in browsers, since RTSP is not natively supported by web applications.

The Video API comes into play by consuming the video segments created by the Cameras API. It processes this data and ensures a secure and scalable storage in the Message Storage System.

The Message Storage System is responsible for preserving recorded videos, allowing users to access them later. Its architecture ensures that data is available in a reliable and organized manner, meeting the needs for retrieval and playback of stored videos. These components work together in integration to offer a robust and efficient video monitoring and recording solution.

The partitioning of the workflow into parallel tasks, such as real-time streaming and recording, ensures that the system supports both the need for live monitoring and the retrieval of stored recordings. Efficient interaction between microservices ensures a stable user experience, even under varying network conditions, while the use of modern protocols such as WebRTC and HLS optimizes the system compatibility and adaptability. This architecture is ideal for scenarios that require high reliability in video management, such as real-time security and surveillance.

The sensor data was from two different sources, as shown in FIG. 8. The first was the smartwatch app (1) that is responsible for collecting biometric data, such as the user's heart rate and geolocation. Such data is sent continuously, at every second, to the Mission API microservice (2) via a WebSocket connection (3), ensuring real-time transmission that is essential for constant monitoring. The second set of data is from the gas sensor (4), which measures concentrations of potentially hazardous gases in the environment. Such devices are mediated by the Data Receiver (5), requiring the implementation of a proprietary protocol for data transmission, sending measurements via Leans (6) and using P2P (Peer to Peer) technology (7) to enable direct communication between network nodes. The Received Data Adapter microservice (8) acts as a centralization point, using the Modbus RTU protocol (9) over RS-485 (10) to receive, organize information and convert the received values into a format compatible with the Mission API.

Next, the measurements are sent via WebSocket (11) to the Mission API (2), ensuring that the information is quickly integrated into the system. The Mission API plays a central role in the architecture, being responsible for consolidating information from both the smartwatch and the Data Receiver.

After receiving the data, the Mission API stores it in a relational database (12), ensuring its organization and persistence for later analysis. In addition, the Mission API uses a Data Cache (13) as an in-memory storage layer, which speeds up processing. The high-performance, cached query was defined to provide efficiency and optimization of resources. This tool is used to temporarily store the results of database queries, allowing faster and more efficient access to the information needed to process sensor measurements.

Once the data is retrieved from the database, it can be presented to users in various formats, such as charts, tables, or reports, making it easier to analyze and interpret the information. This allows users to closely monitor and track then environmental conditions and device performance in real time, as well as perform retrospective analyses to identify trends and patterns over time.

The data flow in the architecture is designed to ensure efficiency and reliability. The smartwatch and gas sensor communicate continuously with the Mission API, which centralizes and processes the information in an integrated manner. The data is stored in the Database for future queries and in the Data Cache to support high-speed operations. Such architecture, by combining smart devices, receivers, and a robust infrastructure of APIs and databases, ensures an effective monitoring and quick responses to emergencies. Furthermore, data redundancy and organization ensure that the system is both reliable and efficient, even in high-demand scenarios.

An operation involves the allocation of resources, planning, and execution of work in a confined space. To start an operation, specific documents are required to ensure the bureaucratic validation of compliance with all safety measures and to ensure that working conditions are aligned with the requirements of the work in question.

The operation is monitored through real-time tracking of devices allocated to the service, including cameras, sensors, and wearable devices. It is also possible to revisit the recording of past operations for analysis and review of previous events. Prior to creation of the operation, the devices and users to be allocated to the operation must already be registered on the platform, including the devices and users with their respective profiles and permissions.

During creation of an operation, the operation's metadata must be provided, such as type, unit of execution, and other relevant details. In addition, a checklist of the necessary documents for the activity should be carried out, such as risk analysis, work permit, among others; a list of extra equipment taken to the operation and which users will be involved should be specified.

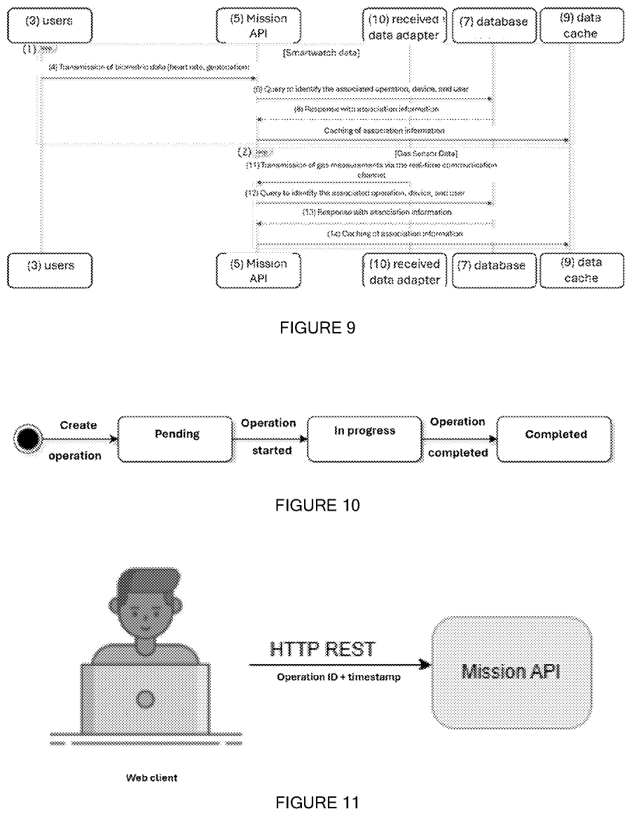

FIG. 9 presents a sequence diagram detailing the flow of information and operations related to real-time sensor monitoring and associated data processing, involving the following main components: the User, the Mission API, the Received Data Adapter, the Database, and the Data Cache. The diagram is structured in two main contexts represented in loops: processing of smartwatch data (1) and processing of gas sensor data (2), both starting with the User's login (3) to the system.

For the first loop, the data source is the smartwatch app, which sends biometric data (4), such as heart rate and geolocation, to the Mission API (5), which acts as the core of the system, coordinating the interactions between the devices and the storage components.

After receiving the data, the Mission API performs a query to identify the operation, the connected devices and associated users (6) in the system's Database (7). Then, the Mission API receives the Response of the association information (8). In addition, the Mission API uses a Data Cache (9) as an in-memory storage layer, which speeds up processing.

In the second loop relating to the processing of gas sensor data, the Mission API (5) communicates with the Received Data Adapter (10) which sends the gas measurements through the real-time communication channel (11). The Mission API is also responsible for querying the database to identify operations, devices and users associated with the mission (12), receiving the Response with association information (13).

Next, the processed data is sent to the Association Information Cache (14) in the Data Cache (9), which acts as an in-memory buffer to ensure efficient real-time processing. This flow occurs continuously, as indicated by the loop in the diagram, demonstrating the continuous collection and synchronization of sensor data.

After the initial data processing in the Data Cache, the system performs persistence in the relational database, ensuring that the information is stored in an organized and reliable manner. The Mission API is responsible for managing this transition, transferring data from the Data Cache to the Database. This process ensures that the collected data is preserved for future analysis and on-demand queries.

In the in both loops of the persistence flow, the database acts as the main repository, storing structured information ready to be retrieved according to the user's needs. As in the previous flow, the loop indicates the continuity of the process, ensuring that the sensor data is stored consistently.

The user, in turn, interacts directly with the system through the Mission API, requesting data or viewing processed information. Requests can include specific queries about sensor readings, active devices, or historical data stored in the database. The Mission API responds to these requests by querying data stored in the database or processing information in real time from the Data Cache, depending on the nature of the request.

Efficient integration between system components, with the Mission API playing a central role in data communication and management. Data cache ensures fast processing of information in real time, while the database guarantees the persistence and organization of data for later analysis. Separating the stages into distinct flows (collection/processing and persistence) demonstrates the robustness and scalability of the architecture, allowing the system to handle large volumes of sensor data efficiently.

FIG. 10 presents a state diagram describing the life cycle of an operation in the system, detailing the transitions between the different states from its creation to its completion. The flow is organized sequentially, indicating the events that lead to each change of state.

The cycle begins with creation of the operation, which automatically enters the “Pending” state. In this state, the operation has not yet begun and is awaiting actions, such as validations or preparations, before execution can begin. During this stage, the final validation of operational documents takes place to ensure compliance with safety requirements. In addition, mobile devices, such as body cameras, smartwatches, and gas sensors, are assigned to the respective users who will participate in the operation.

When initiated, the operation transitions to the “In progress” state, becoming active. At this stage, the defined processes, such as data collection and real-time monitoring, are executed. Upon achieving the objectives or completing the planned stages, the operation transitions to the “Completed” state. At this point, the data collection processes are completed, and the system changes the view from live mode to replay mode, allowing for retrospective analysis. This final state indicates that the operation was successfully completed, with results available for consultation, reporting, and continuous learning.

Transitions between states are triggered by specific events, such as “operation started,” which moves the operation from “Pending” to “In progress,” and “operation finished,” which takes it from “In progress” to “Completed.” This clear and straightforward structure ensures predictability in managing the lifecycle of operations, facilitating the monitoring and control of each stage.

For the monitoring process, the Mission API microservice includes a specific HTTP REST implementation for the sensor data stream, allowing the retrieval of a data range from a specific point in time during a given operation by providing only the operation identifier and the specific timestamp to be queried. This approach, shown in FIG. 11, was designed to simplify the process of obtaining sensor data, allowing users to easily retrieve the desired information for analysis and monitoring during or after completion of the operation.

The process begins with the web client, which can be a browser or a user interface connected to the monitoring platform. The client sends an HTTP REST request to the Mission API containing two main elements: the operation ID, which uniquely identifies the operation in progress or being registered, and the timestamp, which indicates the exact moment the request was made. This information is essential for tracking and recording events related to system operation.

In the Mission API, the received data is processed and integrated with existing records, allowing the system to update the status of operations or store events in a database for future queries. This method ensures that interactions between the client and the system are quick, reliable, and properly organized, facilitating the traceability and analysis of operational information.

Using the HTTP REST standard makes communication efficient and compatible with different devices and systems, while the Mission API centralizes the processing logic, ensuring that operations are managed consistently and securely. This model is ideal for web-based systems, where it is essential to handle requests in a synchronous and standardized manner.

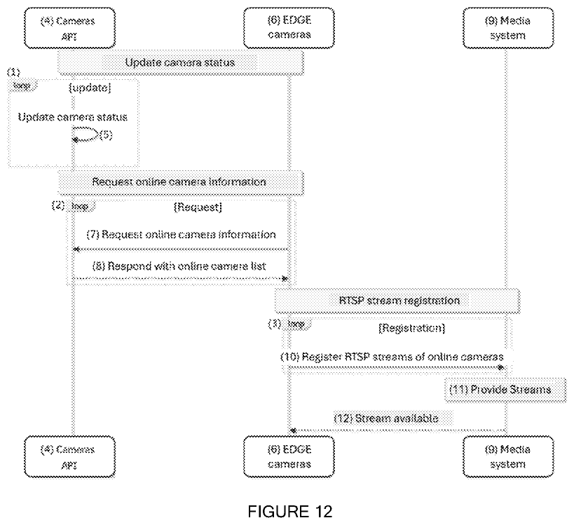

FIG. 12 shows a sequence diagram detailing the flow of interaction between the system components responsible for monitoring and managing cameras: Cameras API, Cameras EDGE, and the Media System, and the process is structured in three main steps, represented in loops: updating camera status (1), requesting information about online cameras (2), and registering RTSP streams (3).

More specifically, the process starts with the Cameras API (4), which performs the status update of connected cameras (5). This status determines whether each camera is online or offline. The process takes place within a continuous loop (1), ensuring that information about the camera status is always up to date. This is essential to identify which devices are active and available for transmission.

In the second loop concerning the camera status, Cameras EDGE (6) requests information about the devices (7) from the Cameras API (4), which responds with the list of online cameras (8). This step also takes place in a continuous loop, ensuring that information about online devices is constantly updated and synchronized across components.

Based on the list of online cameras, the third loop begins, which refers to the recording of RTSP (RealTime Streaming Protocol) streams from the cameras connected to the Media System (9). Then, Cameras EDGE (6) records the RTSP streams from the online cameras (10) in the Media System (9). The Media System is an intermediary server responsible for managing these streams and converting them to the WebRTC format, as it is a protocol compatible with browsers, allowing access to real-time video through web interfaces. Thus, the Media System (9) provides the streams (11) which, once available, can be accessed by end users for real-time monitoring (12).

Separating the content into stages ensures scalability and organization, while using WebRTC optimizes content accessibility across different devices and browsers. This architecture is ideal for monitoring systems that require high performance and constant updates on the status of connected devices.

The WebRTC protocol used for live monitoring was chosen due to the essential requirement of minimizing delay during real-time monitoring. It is the most suitable streaming protocol for web applications that require low latency, providing a smoother and more responsive real-time viewing experience for users.

Live camera monitoring was boosted by the integration between the Cameras API, Cameras EDGE, and Media System services. In general, the Cameras API (4) regularly updates the online and offline status of cameras by accessing these devices and checking their availability on the network. Next, Cameras EDGE (6) periodically makes HTTP requests to the Cameras API to obtain information about the online cameras.

Based on this information, Cameras EDGE records the RTSP streams of the online cameras in the Media System (9) which, in turn, provides a WebRTC stream with individual channels for each registered camera, thus allowing live monitoring of the cameras in real time.

To reproduce video recordings, a video on demand (VOD) model using HLS (HTTP Live Streaming) was employed, which involves saving video segments in storage and making the full video available through an M3U8 playlist, which is a text-based file format used to organize and play media content such as audio and video. The storage of segments and the construction of the playlist were assigned to the Video API, which obtains the live segments provided by the HLS stream generated by the Cameras API and stores them using the Message Storage System services.

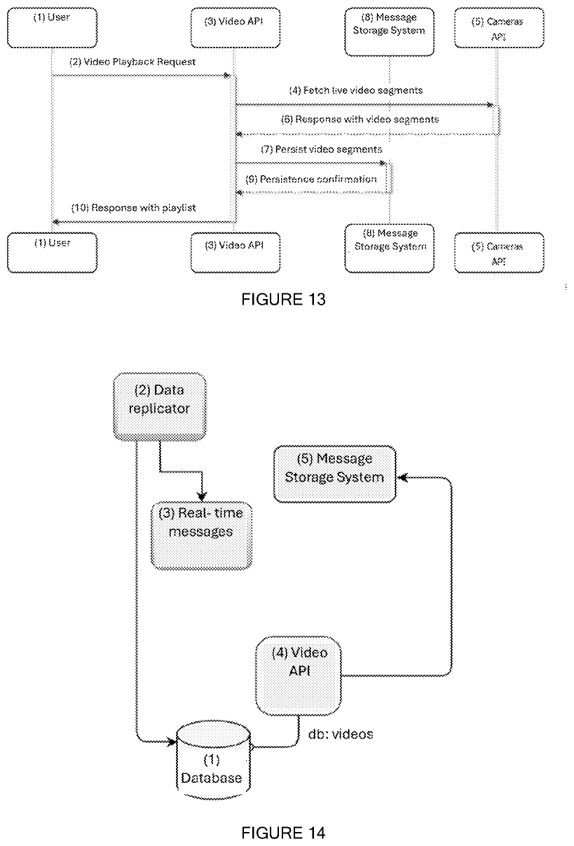

This process is represented in FIG. 13 by a sequence diagram describing the flow of interaction between the system components, including the User, the Video API, the Message Storage System, and the Cameras API, focusing on the request, retrieval, and persistence of video segments. This process is divided into two main stages: the user's request to play video and the data flow to obtain and store video segments.

The flow begins when the User (1) sends a video playback request (2) to the Video API (3) for some specific file. This request can include details such as the video identification or the desired time period. The Video API acts as an intermediary, coordinating access to and delivery of requested video segments.

After receiving the request, the Video API (3) queries (4) the Cameras API (5) to obtain live video segments. The Cameras API then responds by providing the video segments corresponding to the user's request (6).

In parallel, these segments are sent (7) to the Message Storage System (8) to ensure the persistence of new data or updates related to the camera that recorded the video. The Message Storage System, in turn, confirms the persistence of the data and returns this confirmation to the Video API, ensuring that the information is properly recorded and synchronized (9).

With the video segments retrieved and the persistence flow completed, the Video API (3) responds to the user with the HLS playlist URL (10), which allows continuous playback of the videos in adaptive streaming format. The format divided into small segments offers a more stable and responsive viewing experience, even under varying network conditions.

This architecture ensures efficient integration between components, allowing the user to access videos quickly and reliably, while data is managed and stored in an organized and scalable manner. The Video API plays a key role in this process, orchestrating the interactions between the user, the Message Storage System, and the camera data managed by the Cameras API. The combination of HLS and robust persistence makes the system ideal for monitoring and querying recordings in real time or on demand.

Regarding data synchronization, it is important to highlight that it plays an essential role in the context of monitoring of operations, in particular concerning remote access to data outside the operation's local network.

By synchronizing data between the local server and the cloud, the system ensures that the most recent and accurate information is available for viewing and management, regardless of the users' geographic location. This allows remote teams, auditors, or relevant authorities to access and monitor operations in real time, even when they are not physically present on-site.

Such remote access capability facilitates the decision-making and quick action in response to events or instances that require intervention, contributing to an effective and secure management of operations. In summary, synchronizing data between local and cloud servers enables continuous and efficient operation of the monitoring system across a variety of scenarios and conditions.

Two critical synchronization needs were identified for the effective operation of the system: database synchronization and synchronization of video segment files. These needs required specific approaches and appropriate solutions, which function according to the availability of access to the external network.

FIG. 14 schematically presents the synchronization services between on-premise and cloud instances. The architecture that integrates components for capturing changes in the database, event processing and video storage, creating a continuous and synchronized data flow, starts with the Database (1), which stores structured information and video metadata. In this context, the Data Replicator (2) plays a key role as a real-time change capturing tool, monitoring events in the database, such as insertions, updates and deletions, and converting these changes into events for consumption.

The events captured by the Data Replicator are sent to the Real-Time Messaging Service (3), a platform that operates as distributed messaging middleware. The Real-Time Messaging Service ensures that events are reliably delivered to connected services, such as the Video API (4), while offering high scalability and efficiency.

The Video API consumes events from the Real-Time Messaging Service and performs the necessary processing to manage the videos. This includes integrating the received information with the Message Storage System (5), which is designed to preserve video files in a secure and scalable manner.

After processing the data and storing the videos in the Message Storage System, the Video API updates the Database with additional information, such as the status of the videos or new metadata. This continuous feedback loop maintains consistency between the relational database and the video storage system, ensuring that all parts are synchronized.

This architecture provides a robust and efficient solution for managing large volumes of data in real time. The Data Replicator, together with the Real-Time Messaging System, allows for the efficient capture and distribution of changes in the Database, while the Video API manages the processing and interaction with the Message Storage System. Integration of these components ensures the reliability, scalability, and accessibility of the system, which is ideal for applications that require continuous monitoring and quick processing of information.

The Data Replicator, as a Change Data Capture (CDC) tool, integrates directly with databases to capture data changes in real time. This solution monitors the database change log (such as the binary log) to detect and record insert, update, and delete operations.

When there is a change in the local database, the Data Replicator captures this change and transforms it into a structured event, such as a JSON (JavaScript Object Notation) message. These events are then published to a Real-Time Messaging System topic, ensuring reliable message delivery and enabling asynchronous, real-time processing of data changes.

Regarding the cloud server, a consumer of the Real-Time Messaging System receives these events and applies them to the remote database, keeping the two databases synchronized. The bucket replication feature in the Message Storage System allowed data stored in one bucket to be automatically replicated to another bucket on a different storage server. When a new video segment is stored in the local Message Storage System by the Video API microservice, the bucket replication functionality is triggered. The local Message Storage System, in turn, is responsible for automatically replicating video segments to the Message Storage System hosted on the cloud server, ensuring that the data is always available in both instances.

To enable live video monitoring with WebRTC and expose a WebRTC server to an external network, it is paramount to adhere to certain information security measures. This includes the proper configuration of firewalls, the use of robust encryption protocols, and the implementation of effective authentication and authorization to ensure the integrity and confidentiality of transmitted data.

Furthermore, in the current microservice implementation, there is a dependency between the Mission API and the Cameras API. However, the Cameras API operates exclusively on the on-premise instance and not on the cloud. To resolve this issue, a specific redesign of the communication architecture between microservices was necessary, aiming for a more flexible and adaptable approach that allows for an efficient operation in both environments, ensuring that the services function harmoniously and without unwanted dependencies.

The main challenge lies in creating an integration platform capable of handling a wide variety of heterogeneous devices. Therefore, a flexible and robust architecture was developed, which is capable of supporting an effective communication and interaction between different types of devices, such as cameras, sensors, and wearable devices, each with its own specific protocols and requirements.

The proposed solution ensures that this platform is scalable, secure, and easy to maintain, allowing for continuous expansion and adaptation to meet the demands of monitoring operations in confined spaces. It is worth noting that these limitations are a result of the initial design of the monitoring system, which was developed primarily for the on-premise (portable) instance, without fully considering the requirements and challenges of the cloud environment.

### System Navigation Menu

The web platform was developed using React v18, the latest major version of the React JavaScript library, based on the ECMA (European Computer Manufacturers Association) Script 6 (TypeScript) specification, together with styling implemented in Vanilla CSS (Cascading Style Sheets). To manage project dependencies, NPM (Node Package Manager) was used as the package manager and online repository for publishing open-source projects, and Webpack was employed as the bundler (output file) to organize and pack the project files.

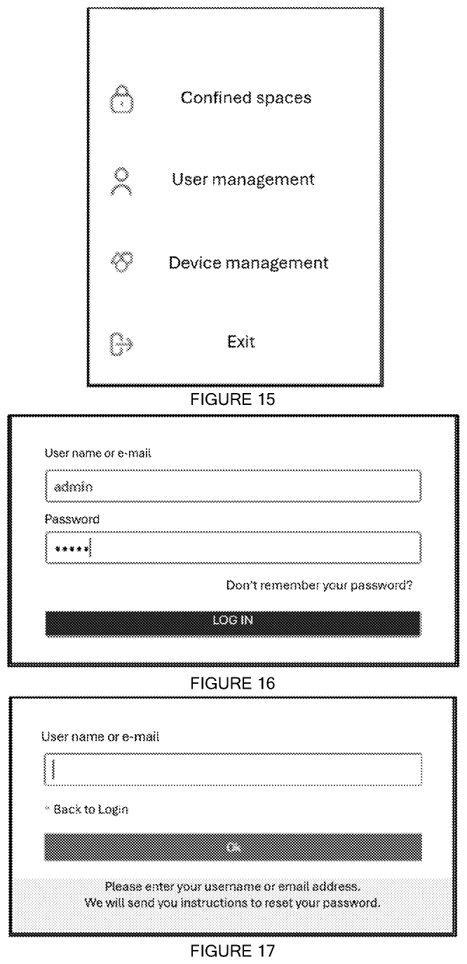

FIG. 15 shows the navigation menu developed for the platform. After logging in, the user may choose to access the following icons: “Confined spaces”, “User management”, “Device management” or “Exit”.

### User Management

Authentication on the web platform is performed by the keycloak-js library, which communicates directly with the system authentication server. Such integration allows users to authenticate, receiving an access token that validates their permissions and authorizes access to the system APIs. FIG. 16 shows the authenticator customized user login screen for the platform, and FIG. 17 shows the authenticator customized password reset screen.

On the User Management screen, administrators have full control over the management of users registered in the system. They can create user profiles, assign specific roles, and adjust access permissions as needed. In addition, the screen offers an intuitive interface for editing and deleting existing users, providing flexible control over who can access and interact with different parts of the system.

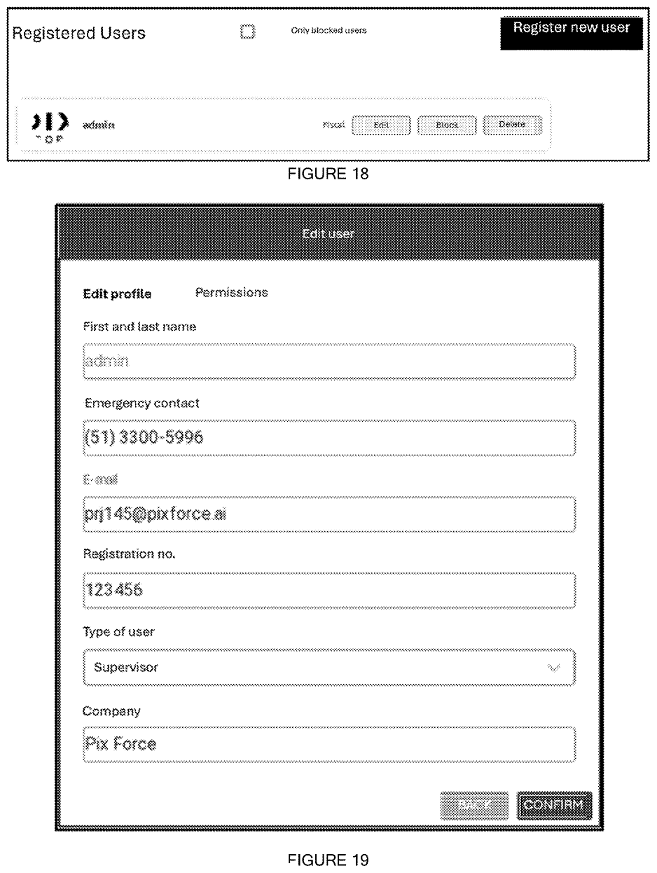

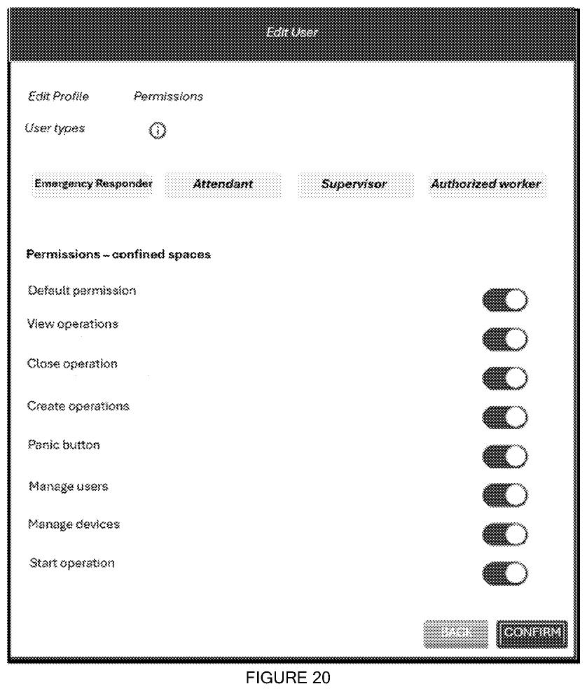

FIG. 18 shows the user management screen and the options available for editing users already registered in the system and for creating new users. By choosing to edit an existing user's profile, a dialog box (pop-up) is displayed, as shown in FIG. 19, allowing both the modification of the worker's personal data, as well as the user type and assigned permissions. By clicking on “Permissions”, still on the editing screen, a dialog box (pop-up) is displayed, represented in FIG. 20, where it is possible permissions for a specific user can be deleted or added, regardless of their role.

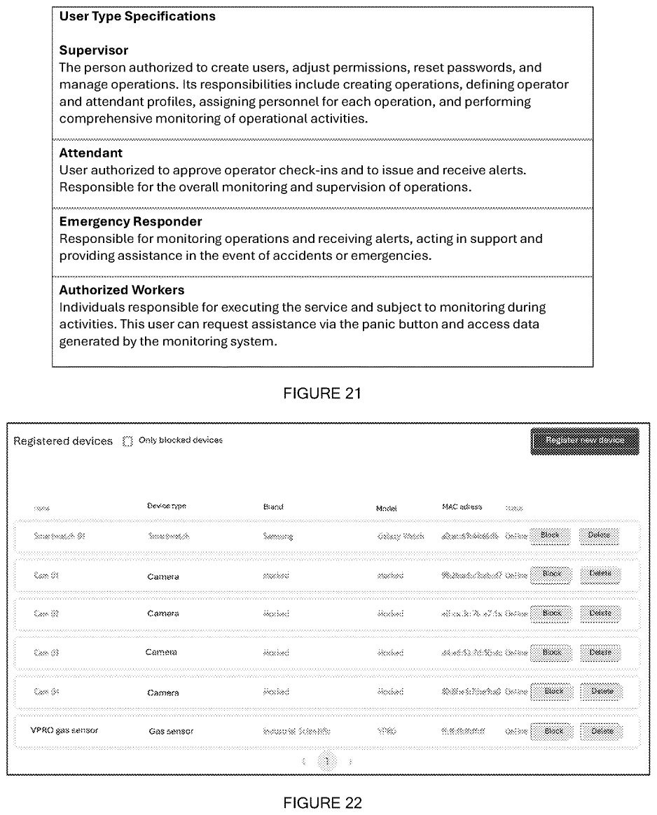

By clicking the information icon next to “user types” on the Permissions screen, a dialog box (pop-up) is displayed with the specifications for each type of user, as shown in FIG. 21.

Supervisor: The person authorized to create users, adjust permissions, reset passwords and manage operations. Its responsibilities include creating operations, defining profiles for operators and attendants, assembling the team for each operation, and comprehensively monitoring operational activities.

Attendant: The user authorized to approve operator check-ins and issue/receive alerts. Responsible for the full monitoring of operations.

Emergency responder: Responsible for monitoring operations and receiving alerts, providing support and assistance in case of accidents.

Authorized workers: Individuals in charge of performing the service and subject to monitoring during the activities. This user may request help via the panic button and access the data generated by the monitoring system.

Therefore, the role descriptions and the predefined permissions for each user type can be viewed.

### Device Management

The Device Management screen, accessed via a specific link in the platform menu, focuses on managing the devices used/registered in the system. Users may add new devices, configure specific settings, and update information for existing devices. This screen provides resources to monitor the status and connectivity, in addition to being able to block or delete specific equipment, allowing for efficient management of the system device infrastructure.

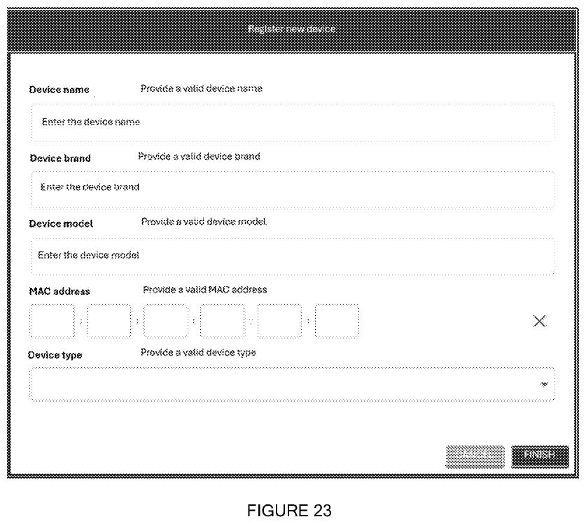

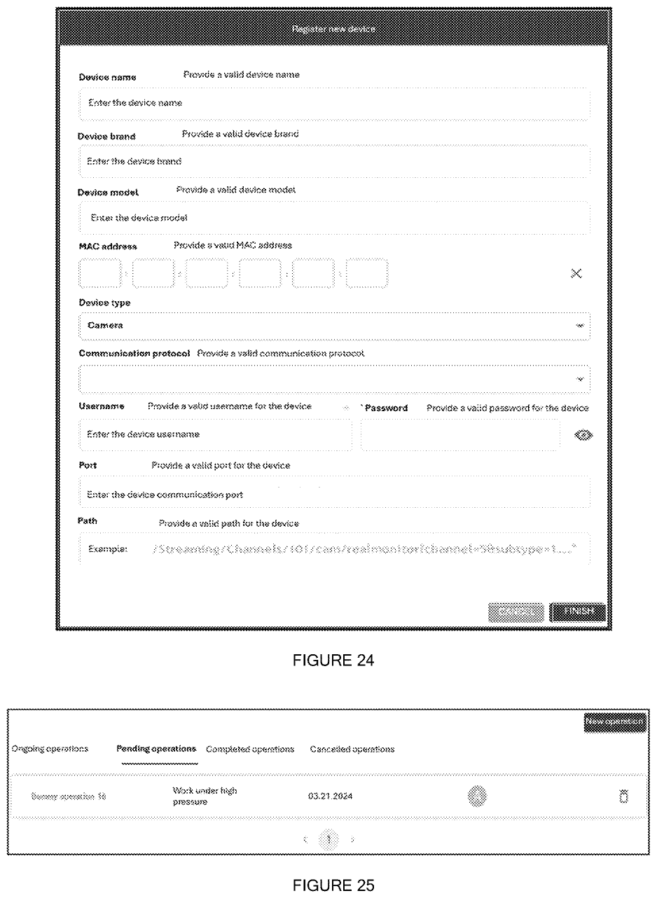

FIG. 22 shows the screen with an overview of the list of devices registered on the platform. When there is a need to create or edit some equipment, a dialog box (pop-up) is displayed, as shown in FIG. 23, which allows the insertion or modification of relevant information. Such information is standard for all devices; however, for imaging devices, in addition to fields such as name, brand, and model, the form also includes specific fields for configuring the properties of the RTSP stream. These specifications may include the communication protocol, username, password, port, and the RTSP stream path, as illustrated in FIG. 24.

These details are essential to ensure that the system can correctly connect to and view images from the cameras in real time, allowing for a customized and precise configuration of the imaging devices. This ensures that all the information necessary for the correct functioning of the cameras is captured and stored properly in the system.

### Operations Management

By accessing the “Confined spaces” option in the platform menu, a screen is displayed that serves as the central point for managing and monitoring operations carried out in confined spaced. Here, users have access to a detailed view of ongoing operations, including real-time information and historical records. The interface provides features for live monitoring, such as viewing cameras and sensor data, allowing for a precise, real-time tracking of ongoing operations. This screen is shown in FIG. 25. The user can choose to view a list of operations that are in progress, pending, completed, or canceled. Additionally, the user may create a new operation or delete existing ones.

The user can also access the operation monitoring screen by selecting that operation on the “confined spaces” screen. Such interface comprises four main views, each organized into distinct tabs for better organization and access to relevant information. These tabs are the following: Live view, Operation data, Deviations and alerts, and Geographic data.

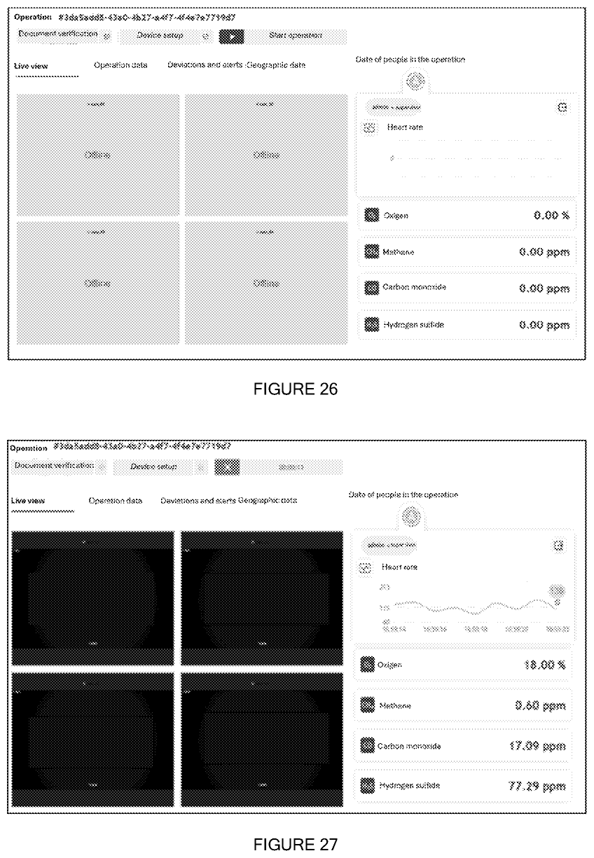

FIG. 26 shows the monitoring dashboard of the “Live View” tab of an operation that is “pending” and FIG. 27 of an operation that is “in progress”. This screen displays information dynamically and in real time from sensors and cameras linked to the ongoing operation.

Users can instantly track the current conditions of the monitored environment, providing a detailed, real-time view of what is happening. It is also possible to monitor the heart rate of the people participating in the operation.

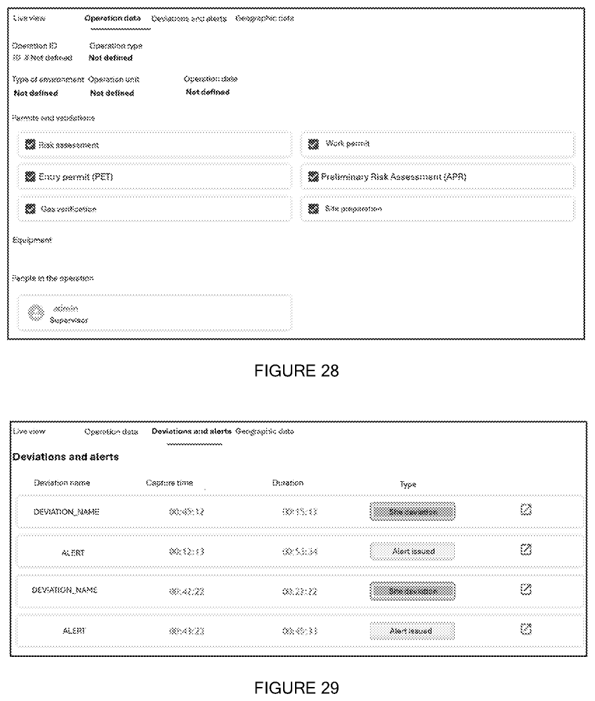

In the “Operation Data” tab, users have access to a comprehensive compilation of data and information provided during the operation registration process. This section provides essential details about the nature of the operation, such as its type, location, and other relevant information that helps contextualize the ongoing monitoring, as seen in FIG. 28.

The third tab, designated as “Deviations and alerts,” provides a detailed list of deviations and alerts identified during monitoring of the operation (FIG. 29). Users can quickly examine any anomalies or non-conforming events detected by the sensors and cameras, allowing for immediate intervention, if necessary.

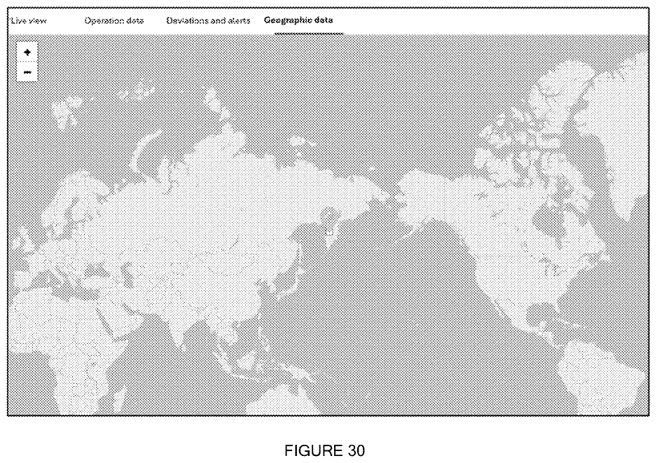

Finally, the “Geographic Data” tab displays an interactive geolocation map with the data collected by the sensors during operation (FIG. 30). This spatial visualization allows users to observe the geographical distribution of the collected information, providing valuable insights into the conditions of the monitored environment and potential patterns or trends.

The Leaflet library was used to create the map view. This open-source JavaScript library provides a flexible and highly customizable way to integrate interactive maps into web applications. Furthermore, to render the maps, tiles from the OpenStreetMap library were used, which provides an open and collaborative data source for detailed and up-to-date maps.

Depending on the monitoring mode selected (live or recordings), the interface offers specific features. In the case of live monitoring, users have access exclusively to a real-time view of the operation information and data. On the other hand, in recording monitoring, a timeline cursor is available that allows navigation through the complete replay of the operation data, including videos and sensor information.

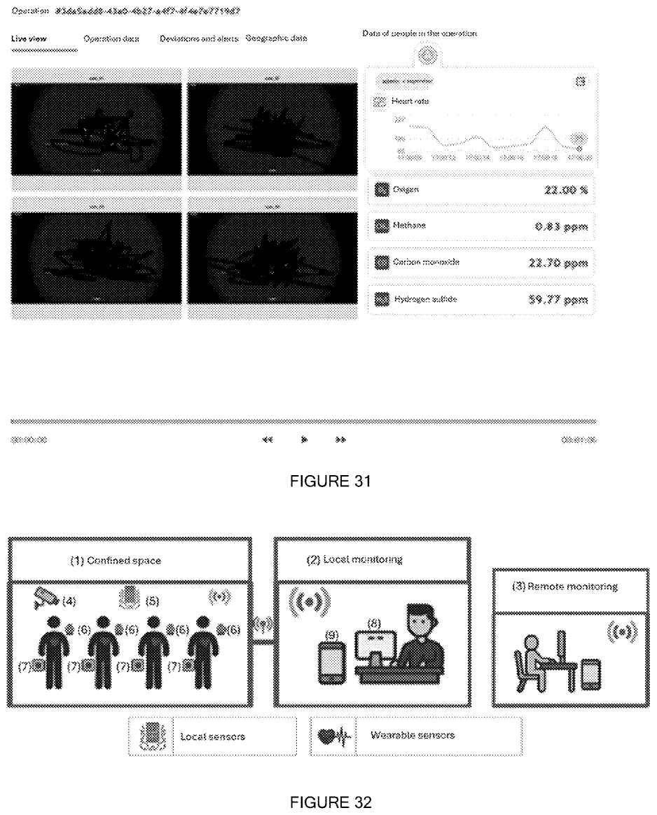

This feature offers a detailed, retrospective analysis of activities and conditions during operation, facilitating review and informed decision-making. FIG. 31 shows the monitoring dashboard of a generic recorded operation, with information about the environment and employees involved, according to the period selected in the timeline.

Finally, in another embodiment of the present invention, a non-transient, computer-readable medium is provided. This medium may include, for example, a memory, a flash memory, a hard disk, a compact disk, or any other device capable of storing computer instructions. When read by a computer, said medium enables the equipment to execute the computer-implemented method disclosed in the present invention.

### Overview of the Computer-Implemented Method for Integrated Monitoring of Activities in Confined Spaces

An overview of the Computer-Implemented Method for Integrated Monitoring of Hazardous Activities in Confined Spaces is illustrated in FIG. 32. The method consists of three main components: (1) Confined Space, (2) Local Monitoring, and (3) Remote Monitoring.

In the confined space, sensors and wearable devices are used to capture data in real time. These devices include surveillance cameras (4) for visual monitoring, gas sensors (5) for environmental analysis and wearable devices such as smartwatches (6) that measure biometric data such as heart rate and body cameras (7).

In Local Monitoring (2), the data captured in the confined space is transmitted to a local control station, where operators monitor the information in real time through screens (8) and mobile devices (9), allowing for the quick identification of alerts or critical events. The representation indicates the availability of Wi-Fi signal both inside and outside the confined space.

Finally, in Remote Monitoring (3), data is made available to teams at remote locations, allowing managers or auditors to monitor the operation via the internet, using computers or mobile devices connected to the network. This method ensures that the operation is monitored accurately and safely, regardless of the operators' physical location.

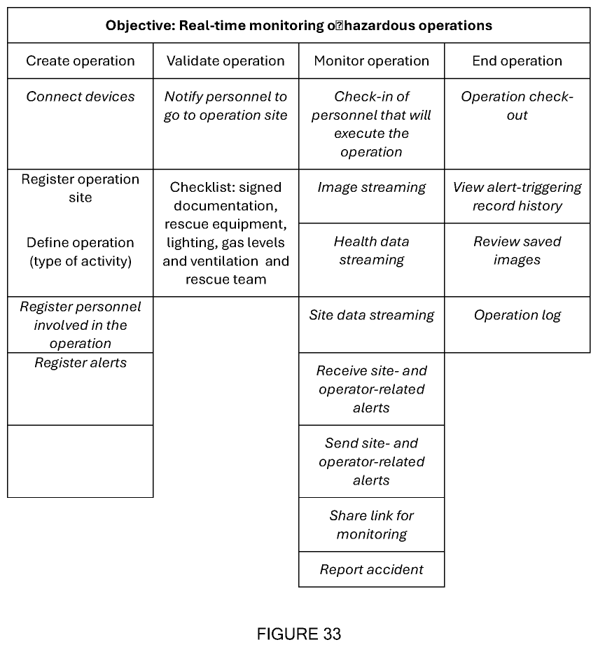

FIG. 33 schematically represents the workflow organized in steps for real-time monitoring of a hazardous activity in a confined space using the computer-implemented method described hereinabove. The flow is divided into four main phases: Create the Operation, Validate the Operation, Monitor the Operation, and Terminate the Operation, each with specific tasks that ensure the safe and efficient execution of the operation.

To create operations, initial configuration actions are performed. Users connect devices to the system, register the operation location, and define the type of activity to be performed. In addition, the people involved in the operation are registered in the system, and specific alerts are configured to monitor potential risks. Finally, all documents relevant to the operation, such as authorizations and procedures, are included.

Next, the validation step involves verifying the preparations before the operation starts. To that end, notifications To this end, notifications are sent to the involved parties requesting their presence at the activity site, and a checklist is performed to confirm that the documentation has been signed and that all essential resources, such as rescue equipment, lighting, ventilation, and gas monitoring, are available. This validation ensures that safety conditions are met before the operation starts.

The next step involves monitoring the operation. During the operation, the system allows real-time monitoring of data from various sources, including the status the involved parties (check-in), streaming images captured by cameras, operator health data (such as heart rate), and environmental information, such as gas levels. In addition, alerts related to the environment or operators are received and, when necessary, sent to notify critical events. The monitoring phase also allows for the sharing of links for remote monitoring and the immediate reporting of accidents.

In the final phase, the operation is concluded with a checkout process for the participants. The system allows users to view the records that generated alerts and the images captured during the operation. A final report is generated, consolidating the operational information for further analysis and learning, including data on alerts, environmental conditions, and operational performance.

The computer-implemented method developed herein was structured and automated to manage and monitor operations in hazardous environments, where each phase is essential to ensure that risks are mitigated and that operations are carried out safely and efficiently. Integration of connected devices, real-time data collection, rigorous validation, and final analysis highlight the innovation in managing activities in confined spaces, aligned with the goals described in the patent document.

### Test Results

The proposed solution used a modular approach that provided for a flexible integration with devices from different manufacturers. This architecture facilitates maintenance and scalability of the system, despite initial challenges with vendors and streaming stability. The tests were carried out in two real-world scenarios.

The tests performed on the proposed solution followed three distinct steps: laboratory testing and two tests in real environments. Laboratory tests were carried out in a controlled environment in order to validate system components, identify potential failures, and adjust functionalities before application on the field. These tests focused on evaluating the device performance, microservice integration, and efficiency in data collection, transmission, processing, and storage.

The test environment was set to simulate typical operating conditions, including devices such as cameras, gas sensors, and smartwatches. For the video system evaluation, cameras were configured to transmit streams in the RTSP protocol, which were recorded and processed by Cameras EDGE. These streams were relayed by the Media System, where they were converted to WebRTC format, ensuring compatibility with web browsers. The average transmission delay was measured using specific latency monitoring tools, and the results showed consistent times within expectations for a controlled environment, allowing for the identification of communication bottlenecks.

The gas sensors were tested in simulated scenarios by varying the gas concentrations to verify the accuracy of the measurements. This data was sent to the Data Receiver, where it was processed using the Modbus RTU protocol and transmitted to the Mission API via WebSocket. The system was assessed for its ability to respond to rapid changes in sensor values, ensuring that measurements were correctly recorded in the database.

The smartwatches were tested in scenarios that simulated movement and physical exertion, monitoring heart rate and location. This data was transmitted to the Mission API at one-second intervals, and its accuracy was compared to reference devices, demonstrating high reliability. The panic button on smartwatches was tested to ensure that alerts were sent to the system and processed without significant delays.

In addition, simulations of critical scenarios were carried out, such as a communication failure of the device or the momentary disconnection of a sensor. The system was assessed for its ability to automatically reconnect and preserve data during interruption, using the Data Cache as temporary storage. These simulations confirmed that the system was resilient and capable of maintaining integrity of information even under adverse conditions.

In processing video data, the Video API was tested to verify its efficiency in consuming HLS segments generated by the Cameras API and in securely storing them in the Message Storage System. Load tests were carried out to assess the system performance in scenarios with multiple cameras transmitting simultaneously, ensuring that the system could handle the expected data volume without significant performance degradation.

Communication with the cameras API was successful using WebRTC for live streaming. Communication with body cameras was also successful live using WebRTC. Reception of data from the gas sensors was stable and with no frequent drops, with data received every 20-30 seconds.

Communication with smartwatches proved effective in receiving data such as geolocation and heart rate, sent via websocket every second. The platform worked well in offline mode, allowing the registration of devices, users, and operations, with data synchronization upon restoration of internet connection.

Laboratory testing allowed for the validation of all components under controlled conditions, ensuring that the system was functional and optimized prior to being implemented on the field. These tests highlighted the efficient integration of microservices, the reliability of the devices, and the robustness of the architecture in managing real-time data. The information gathered was essential for refining performance of the system and ensuring its effectiveness under real-world operating conditions.

The first real-world test was carried out at the REFAP Refinery, located in Canoas, RS, Brazil In this test, we monitored a service being performed in a confined space inside a tank. Communication between the devices and the system was efficient, with data being transmitted and received as expected, demonstrating the robustness of the solution in an industrial environment.

The tests carried out at REFAP refinery aimed to evaluate the system performance and effectiveness in a real industrial environment, focusing on device integration, real-time data transmission, and the functionality under demanding operating conditions. The test structure was designed to assess the behavior of system components, including cameras, gas sensors, and wearable devices, and to validate the reliability of the flow of information between the different modules.

The system was installed in an operational area of the refinery, with devices strategically distributed to monitor activities in confined spaces and higher-risk areas. Cameras were configured to capture videos using the RTSP protocol and send the streams to the server, where they were processed by the Cameras API and retransmitted in WebRTC format by the Media System. Simultaneously, gas sensors were deployed for environmental monitoring, and operators used smartwatches to send biometric data, such as heart rate and location, at one-second intervals, connected to the system via WebSocket.

Transmission and storage of data were performed via microservices configured on the server. The Mission API received data from sensors and wearable devices, processing the information in real time and storing it in the database. The video system managed by the Video API was integrated with the Message Storage System to ensure the secure storage of video segments, allowing for later queries and playback.

The results were analyzed based on criteria such as data transmission latency, video stream quality, and measurement accuracy. During testing, an average delay of 30 seconds was observed in the transmission of video streams, mainly due to the retransmission process by the Media System and the local network infrastructure, which was identified as an area for improvement. Data quality from the sensors and biometric devices was assessed by comparing it to reference standards, demonstrating high accuracy and consistency of the information collected.

Furthermore, the system was subjected to simulation scenarios, such as the detection of critical conditions with gas sensors and alerts generated by the panic button on smartwatches. These tests confirmed that the system was capable of processing and responding to events in real time, efficiently transmitting alerts to the operators in charge.

The tests at REFAP were essential to validate the robustness and functionality of the solution in an industrial environment. They also provided important insights for optimizing system performance, such as reducing latency in video streams and improving communication in high-volume data scenarios. The assessment has demonstrated the system applicability in refineries and similar industrial environments, highlighting its ability to monitor and manage operations with precision and reliability.

The second real-world test was carried out in a more challenging setting at the RECAP Refinery in Mauá, SP, Brazil. The monitoring was carried out in a heat exchanger, where the work took place in an elevated and difficult-to-access space. Despite the additional challenges posed by this environment, the solution successfully monitored the system, demonstrating its adaptability and efficiency under different operating conditions.

The tests carried out at the RECAP Refinery aimed to validate the functionality and efficiency of the solution developed for real-time monitoring of activities, in particular in critical environments such as confined spaces. These tests were structured in stages to evaluate different aspects of the system, such as data collection, processing, transmission, and usability under real operating conditions.

Initially, the system was deployed in a specific area of the refinery, where devices such as cameras, gas sensors, and smartwatches were installed to monitor operators. The cameras were strategically placed to cover critical areas, while the gas sensors were configured to measure concentrations in sensitive locations, such as enclosed spaces. The operators used smartwatches for biometric monitoring, including heart rate and location.

The devices were connected to the portable server that hosted the system microservices, including the Mission API, Cameras API, and Video API, ensuring that the collected data was processed in real time. The video streams were transmitted using cameras configured to send data via the RTSP protocol, which was converted to WebRTC for playback in web browsers. Simultaneously, data from the gas sensors and smartwatches were sent via WebSocket to the server, where they were processed and stored in the database.

To measure the system effectiveness, two types of analyses were used: response time and data quality. Response time was assessed by monitoring latency from data collection to its visualization by operators on the web interface. In the tests, a delay of approximately 30 seconds was observed in the video transmission, which was identified as an area for improvement. Data quality was verified by comparing the sensor readings with reference equipment installed at the refinery, ensuring the accuracy of the measurements.

The results achieved demonstrated that the system was able to collect and process data in real time, with a high degree of reliability, even in a complex operating environment. Furthermore, integration between the different devices and microservices proved to be robust, allowing for continuous synchronization of information. The tests also highlighted opportunities for improvement, such as reducing delay in video transmission and enhancing the communication between devices and the server.

These tests at RECAP were essential in validating the system applicability under real-world conditions, providing valuable data for future adjustments and improvements, as well as demonstrating that it is possible to implement the solution in other industrial units.

### Examples—State of the Art

Monitoring activities in confined spaces without an integrated system involves manual and decentralized steps. Preparing the environment includes risk assessment, equipment inspection, and verification of safety standards, as well as the formation of a rescue team.

The process for approving the work and mobilizing the team is coordinated, but without a centralized platform. During operation, safety of the workers is monitored by attendants and auditors. Any problems or irregularities are reported via radio or approved devices, which may cause delays.

In the event of an accident, communication and response rely on traditional methods, such as phone calls or radios, making an efficient and quick coordination of responses difficult. Therefore, monitoring without an integrated system is fragmented, subject to delays, and relies on manual processes, compromising efficiency and security.

### Examples—Present Invention

With the integrated monitoring system, the flow of operations in confined spaces has demonstrated significant gains in safety and efficiency. During field tests, it was found that the setup time for starting operations can be reduced compared to traditional methods, thanks to the plug-and-play platform, which allows for quick installation and configuration of monitoring devices.

In monitored operation scenarios, the system efficiently collected and centralized sensor data, eliminating the need for multiple separate systems. Tests showed that incident response time can be reduced, since alerts automatically generated by the platform were sent to the rescue team within an average of 5 seconds after the event was detected. Furthermore, managers had remote access to the information, allowing for real-time interventions and improving team coordination.

During laboratory tests, the latency for data transmission from the sensors to the platform was found to be less than 2 seconds, even under simulated high data traffic load conditions. Integration with the cloud ensured that data was available for continuous and organized consultation and analysis. The platform usability was positively evaluated by the operators, who reported ease of navigation and understanding of the system.

In terms of data collection, the platform demonstrated high efficiency in transmitting data from gas sensors and wearable devices, such as body cameras and smartwatches. In simulated operations, the rate of failure in communication was reduced compared to non-integrated systems.

These results prove that the invention provides a practical and innovative approach to managing operations in confined spaces, with clear benefits in terms of operational efficiency, risk reduction, and usability. The advantages described demonstrate that the solution reliably and effectively meets the demands of the sector.

What is claimed is:

1. An integrated monitoring system for hazardous activities in confined spaces, the system comprising:
a portable machine, comprising a server, on which is hosted a set of microservices deployed independently in Docker containers, wherein each microservice is responsible for a specific role, and wherein the portable machine is configured to be moved to a site where a hazardous activity is performed in a confined space;
a network scanning service, based on an Address Resolution Protocol, configured to identify and manage dynamically connected devices running on the portable machine;
cameras configured to capture first images in real time, the cameras being configured to be connected by Ethernet cables to a switch or directly to a router;
body cameras configured for direct connection to the router;
at least one biometric sensor configured to collect biometric data of at least one workers and transmit the collected biometric data to the server via a WebSocket connection; and
gas sensors configured to measure concentrations of O₂, H₂S, CO and CH₄ and to transmit the measured concentrations to the server using a data receiver and a Modbus Remote Terminal Unit protocol.

2. The system of claim 1, wherein the portable machine is configured to generate autonomous alerts and reports indicating non-conformities and risk situations and to correlate variables to generate data and predict accidents; and
the autonomous alerts are generated by a computer-implemented method based on the collected biometric data, the measured concentrations, the images captured by the cameras, and images captured using the body cameras.

3. The system of claim 1, wherein the portable machine is configured to manage and store the collected biometric data, the measured concentrations, the images captured by the cameras, and images captured using the body cameras and synchronize the collected biometric data, the measured concentrations, and the captured images with the cloud, allowing for queries for audits and investigations.

4. The system of claim 1, wherein the microservices are configured to communicate with each other through a communication protocol; and
the communication occurs synchronously or asynchronously, providing access to a same instance of a database query language for data storage and retrieval.

5. The system of claim 1, wherein the portable machine has at least two network interfaces;
one of the at least two network interfaces is an Ethernet interface for a local network using a computer-implemented method, connected to a local addressing switch, used with the devices and with addressing managed by the local DHCP service; and
another of the at least two network interfaces is for external connectivity with an online service, represented by 4G/5G connectivity.

6. The system of claim 1, wherein the portable machine is configured to manage services through container isolation, using Docker and Docker Compose.

7. The system of claim 1, wherein the portable machine has a USB RS-485 modbus converter connected to a data receiver along with the gas sensors.

8. The system of claim 1, further comprising a Network Time Protocol server configured to synchronize clocks of all devices on a network including the server and the Network Time Protocol server.

9. The system of claim 1, wherein detected network events are configured to published on a RabbitMQ messaging service.

10. The system of claim 1, wherein the cameras and the body cameras are configured to provide Real Time Streaming Protocol stream for video transmission.

11. The system of claim 1, wherein the specific roles of the microservices include mission management, user authentication, video processing, and sensor data collection.

12. The system of claim 1, wherein a user authentication process is configured to take place on the server in which a user submits credentials, and the server is configured to verify the credentials and generate a temporary access token if the credentials are verified.

13. The system of claim 1, wherein users receive permissions on the server according to roles of the users, the roles of the users including supervisor, attendant, emergency responder, and authorized user.

14. The system of claim 1, wherein the at least one biometric sensor is configured to be registered and receive a valid Internet Protocol address during interaction in operations.

15. The system of claim 1, wherein, when the at least one biometric sensor attempt to connect to a network including the server, a corresponding media access control (MAC) address is configured to pass through a Dynamic Host Configuration Protocol server configured to check if the MAC address is valid and correctly registered.

16. The system of claim 1, wherein the microservices include:
a Cameras application programming interface (API) microservice responsible for periodically updating an online or offline status of the cameras in a database, wherein the Cameras API microservice is configured to create a hypertext transfer protocol live streaming stream; and
a Cameras EDGE microservice is configured to, based on the online or offline status of the cameras, take on a role of registering a Real Time Streaming Protocol stream on a Media System server.

17. The system of any one of claim 16, wherein the microservices also include a Video API microservice configured to consume a playlist generated by the Cameras API microservice, collecting video segments, and saving the video segments to a Min. io storage service.

18. The system of claim 1, wherein at least one of:
the specific roles include video capture, biometric data processing, and sensor management,
the cameras are configured to use a protocol for transmission and viewing and the protocol includes at least one of Real Time Streaming Protocol and Web Real-Time Communication,
the biometric data comprises heart rate and geolocation of workers,
the transmission of the measured concentrations to the server is configured to ensure integration of environmental information,
the microservices are configured to communicate with each other through a communication protocol that includes one of: hypertext transfer protocol/representational state transfer, WebSocket, or asynchronous messages,
a user authentication process is configured to take place on the server in which a user submits credentials, the credentials include a username and password, and the server is configured to verify the credentials and generate a temporary access token if the credentials are verified, or
the at least one biometric sensor comprises at least one smart watch.

19. A computer-implemented method for integrated monitoring of hazardous activities in confined spaces using the system of claim 1, of the method comprising:
using the portable machine at the site with the hazardous activity being performed in the confined space;
collecting real-time data on a progress of the hazardous activity in the confined space using the cameras, the body cameras, the at least one biometric sensor, and the gas sensors;
sending the collected real-time data so that monitoring can be performed; and
presenting the collected real-time data to at least one user.

20. The method of claim 19, wherein the collected real-time data is presented to the at least one user in a variety of formats.

21. The method of claim 20, wherein the formats include at least two of charts, tables, or reports.

22. A computer-readable non-transitory storage medium comprising instructions stored thereon, wherein the instructions, when read by a computer, cause the computer to run method of claim 19.
